(19) United States
(12) Patent Application Publication
(10) Pub. No.: US 2015/0011411 A1
(43) Pub. Date: Jan. 8, 2015

(54) BIOMARKERS OF CANCER

(71) Applicants: THE REGENTS OF THE UNIVERSITY OF COLORADO a body corporation, Denver, CO (US); THE UNIVERSITY OF VIRGINA PATENT FOUNDATION, Charlottesville, VA (US)

(72) Inventors: Dan Theodorescu, Englewood, CO (US); Alex Baras, Baltimore, MD (US); Steven Smith, Ann Arbor, MI (US)

(21) Appl. No.: 14/366,031

(22) PCT Filed: Dec. 21, 2012
(86) PCT No.: PCT/US2012/071399

§ 371 Date: Jun. 17, 2014

Related U.S. Application Data
(60) Provisional application No. 61/578,873, filed on Dec. 21, 2011.

(51) Int. Cl. C12Q 1/68; G01N 33/50; G01N 33/574

(52) U.S. Cl. CPC C12Q 1/6886; G01N 33/57434; G01N 33/57407; G01N 33/5011; G01N 2800/52; C12Q 2600/136; C12Q 2600/158; C12Q 2600/118
USPC 506/9; 506/16; 506/18

(57) ABSTRACT

The present invention relates to identification of a transcriptional signature of RalA and RalB GTPase proteins that is present in human cancer cells, and methods of treating the cancer, methods of diagnosis of the cancer, methods of determining predisposition to the cancer, methods of monitoring progression/regression of the cancer, methods of assessing efficacy of compositions for treating the cancer, methods of screening compositions for activity in modulating biomarker of the cancer, as well as other methods and assay systems based on the signature.

### FIELD OF THE INVENTION

The present invention generally relates to biomarkers, methods and assay kits for the identification, monitoring and treatment of cancer patients.

### BACKGROUND OF THE INVENTION

In humans, the Ras-like (Ral) GTPases include the homologous paralogs RalA and RalB, which have been implicated in diverse cellular functions (Bodemann and White 2008). A growing body of literature has implicated these GTPases in key cancer phenotypes such as Ras-mediated transformation (Hamad et al 2002, Rangarajan et al 2004). This transformation is dependent specifically on RalA (Lim et al 2005), and may be further regulated by serine phosphorylation (Sablina et al 2007) while other phenotypes such as regulation of cellular motility (Gildea et al 2002, Oxford et al 2005), invasion (Feldmann et al 2010, Lim et al 2006), and metastasis (Wang et al 2010, Wu et al 2010, Yin et al 2007) are attributed to either RalA or RalB, depending on the model system and cancer type evaluated.

Despite these important in vitro and in vivo findings, there is little evidence supporting the biological relevance of Ral in human tumors. Unlike other GTPases, Ral mutations have not been noted in large (Kan et al 2010, Wood et al 2007) or targeted (Smith et al 2007) screens of common cancers. In contrast, overexpression of RalA has been observed in a small number of muscle invasive bladder cancers (MIBCs) (Smith et al 2007), and in advanced forms of prostatic adenocarcinoma (Varambally et al 2005). Neither of these studies evaluated the tumors by in situ technologies such as immunohistochemistry, precluding assessment of expression in distinct tumor compartments.

However, while expression of the GTPase itself contributes to the output of the Ral pathway, factors that impact GTPase activation such as microenvironmental stimuli, post-translational modifications including phosphorylation, and differential expression of downstream Ral downstream effectors (Smith et al 2007) are likely to play significant roles in determining the relevance of Ral expression in cancer (Smith and Theodorescu 2009). Ral GTPases also regulate key transcriptional pathways including transcription through TCF, NF-κB, Stat3, HSF, E2F, and forkhead family transcription factors, ZONAB, and RREB1, reviewed recently (Neel et al 2011). Targets of these pathways have been demonstrated to include key cancer genes such as cyclin DI (Henry et al 2000), VEGFC (Rinaldo et al 2006), and CD24 (Smith et al 2006), supportive of the important role of Ral-dependent transcription in cancers.

Thus there exists a need for the evaluation of the status and clinical relevance of Ral in several human cancers and coupling that with evaluation of the transcriptional output of these proteins as a surrogate of Ral pathway activity. Such an evaluation will provide accurate methods to identify cancers in a patient, as well as their proclivity for metastases/relapse.

### BRIEF DESCRIPTION OF THE DRAWINGS

FIG. 1 shows Expression of RalA and RalB by immunohistochemistry in 110 human urothelial bladder tumors in patients treated by radical cystectomy. A tissue microarray of bladder carcinomas (Smith et al 2009), stages pTa-T4, was stained with antibodies specific for RalA and RalB. A. Representative photomicrographs of strong, diffuse RalA staining (RalA High, solid) and weak RalA staining (RalA Low, dashed). Kaplan-Meier analysis of overall survival, stratified by expression level of RalA, showing a non-significant trend in favor of poorer overall survival for cases expressing strong RalA (Log Rank P=0.16). Wilcoxon-Breslow testing of these curves, which weights early events, identified a significant difference (P=0.04). B. Similar micrographs for RalB, showing strong diffuse staining (blue, solid) and weak RalB staining (blue, dashed). Kaplan-Meier analysis for RalB expression, finding non-significant difference by Log Rank or Wilcoxon-Breslow methods.

FIG. 2 shows the Core Transcriptional Signature of Ral GTPases. A. A signature of Ral consisting of 39 probes was developed from genes regulated 2-fold by RalA and RalB expression in UM-UC-3 cells. Hierarchical cluster analysis of gene expression data for 91 bladder cancers (Sanchez-Carbayo et al 2006) and control siRNA treated UM-UC-3 cells (siControl) (thus, Signature positive) or RalA and RalB-depleted siRNA duplexes (siRal) (Signature negative), showing association of the signature with muscle-invasive tumors. B. Expression of the Ral signature from cases in A. was quantitated by use of a weighted KNN classification algorithm (Smith et al 2011), which outputs a score from 0, Signature negative, to 1, Signature positive. Dotplots show Ral signature scores, with differences in score distributions for non-muscle invasive pTa/T1 cases and muscle invasive pT2+ cases tested by Mann-Whitney methodology and AUC and group medians indicated (black lines). C. Ral signature probes were mapped to a different microarray platform, and Ral transcriptional signature scored for UC cases (N=71) from a prior report (Dyrskjot et al 2003) with analysis by Mann-Whitney Utest. D. Another cohort of bladder tumors (N=30) (Stransky et al 2006) showed similar results to those in B-C.

FIG. 3 shows the association of the Ral Signature Score with experimental and patient outcomes. A. Using a serial metastasis model that we have recently developed and analyzed by microarray (Overdevest et al 2011), we found significantly higher Ral signature scores in metastatic Lul2 cells compared to parental Luc cells. B. Using microarray data from a recent publication where highly tumorigenic/stem cell-like cells were isolated from bladder cancer cells by cell sorting (He et al 2009), we found significantly higher Ral signature scores in highly tumorigenic cells compared to parental or negative sorted cells (Mann-Whitney, plot shows median plus 95% Cl). C. In the Sanchez Carbayo et al. cohort of 91 bladder cancers used in FIG. 2, the Ral signature score was associated with overall survival (K-M plot showing signature score >0.5 positive versus <0.5 negative, Log Rank test). D. In non-muscle invasive tumors, expression of the Ral signature is significantly associated with subsequent progression to muscle invasion in a previously published cohort (N=29) (Dyrskjot et al 2005) (similar plot and Log Rank test to C.).

FIG. 4 shows Ral signature scores in squamous malignancy. A. Using data from a published cohort of 53 patient-matched esophageal SCCs and adjacent normal mucosae (Su et al), we observed a highly significant trend toward lower Ral signature score in cancer (P<0.0001, Wilcoxon matched pairs). Before and after plot shows matched pairs of mucosa and cancer, with decreases in signature score plotted red (N=37), similar scores plotted black (N=13) and increased scores plotted blue (N=3). B. in a second, unmatched cohort of 12 mucosae and 26 oropharyngeal SCCs (Ye et al 2008), a similar significant pattern was identified (Mann-Whitney test, signature scores plotted and medians per group indicated, black lines).

FIG. 5 shows the Ral signature score and prostate cancer disease aggression. A. Using data from a recently published cohort (N=131) of prostatic adenocarcinomas from Taylor et al. (Taylor et al 2010) we found that Ral signature scores could significantly stratify biochemical recurrence free survival. (Kaplan-Meier analysis, Ral signature classes plotted at optimal discriminating point, Log Rank Test). B. In the Taylor et al. cohort, a significant difference was observed between cases that did or did not evince invasion of the seminal vesicle at prostatectomy (Mann-Whitney test, signature scores plotted and medians indicated per group, black lines). C. A similar plot to that in A, but plotted for cases from the Swedish Watchful Waiting Cohort (N=281) recently reported by Sboner et al. (Sboner et al 2010) showing disease specific survival stratified by Ral signature score (Kaplan-Meier analysis and Log Rank test).

FIG. 6 shows that the Ral signature score is sensitive to androgen status in prostate cancer. A. Using published expression profiling data for LNCAP cells treated with control or charcoalstripped (steroid hormone free) medium over a time course of 12 months (D'Antonio et al 2008), we observed significant and durable induction of the Ral signature over time in androgen deprived (charcoal stripped serum, CSS, red) as compared to control full serum treated cells (full serum, blue), Mann-Whitney test. B. Quadruplicate KuCAP-2 (Terada et al 2010) xenografts were analyzed at androgen-dependent baseline (AD), at castration treatment induced growth nadir (Tx), and during androgen independent (Al) regrowth. A significantly higher Ral signature score was seen in treated and androgen independent tumors (Mann-Whitney test of all treated versus baseline replicates, plot shows median plus 95% CI). C. Consistent with the in vitro and in vivo results in A and B, we observed significantly higher Ral signature scores in a published cohort (Best et al 2005) of androgen independent tumors (N=10) as compared to androgen dependent (N=10) cases (Mann Whitney test, signature scores plotted, and medians indicated per group, black lines). D. We also observed a similar higher Ral signature score in androgen independent cases in a second cohort (Wei et al 2007) of androgen independent (N=8) as compared to androgen dependent (N=18) cases.

FIG. 7 or S1 shows antibody specialty of the anti-RalA antibody. A. Indicated siRNAs targeting firefly luciferase (siControl), RalA, or RalB or expression vectors for FLAG (control), FLAGRalA, or FLAG-RalB were transiently transfected into UM-UC-3 bladder cancer cells and lysates immunoblotted for RalA. B. To demonstrate that immunohistochemistry for RalA may detect RalA expression in a semiquantitative manner, GFP (control) and GFPRalA overexpressing (test) cell pellets were formalin-fixed and paraffin embedded. Sections were stained as described, demonstrating a significant difference in stain intensity between the indicated panels. C. Kaplan-Meier analyses and log rank P-value comparing overall survival over time for all cases of bladder carcinomas (including urothelial and non-urothelial cases) by RalA staining class. D. A similar analysis to that of A. restricted to non-urothelial cases. For urothelial cases alone, see FIG. 1B.

FIG. 8 or S2 shows antibody specificity of the anti-RalB antibody. A. To demonstrate antibody specificity, indicated siRNAs targeting firefly-luciferase (siControl), RalA, or RalB or expression vectors for FLAG (control), FLAGRalA, or FLAG-RalB were transiently transfected into UM-UC-3 bladder cancer cells and lysates immunoblotted for RalB as reported. B. To demonstrate that immunohistochemistry for RalB may detect RalB expression in a semiquantitative manner, Flag-control and Flag-RalB overexpressing (test) cell pellets were formalin-fixed and paraffin embedded. Sections were stained as described, demonstrating a significant difference in stain intensity between the indicated panels. C. Kaplan-Meier analyses and log rank Pvalue comparing overall survival over time for all cases of bladder carcinomas (including urothelial and non-urothelial cases) by RalB staining class. D. A similar analysis to that of A. restricted to non-urothelial cases. For urothelial cases alone, see FIG. 1D.

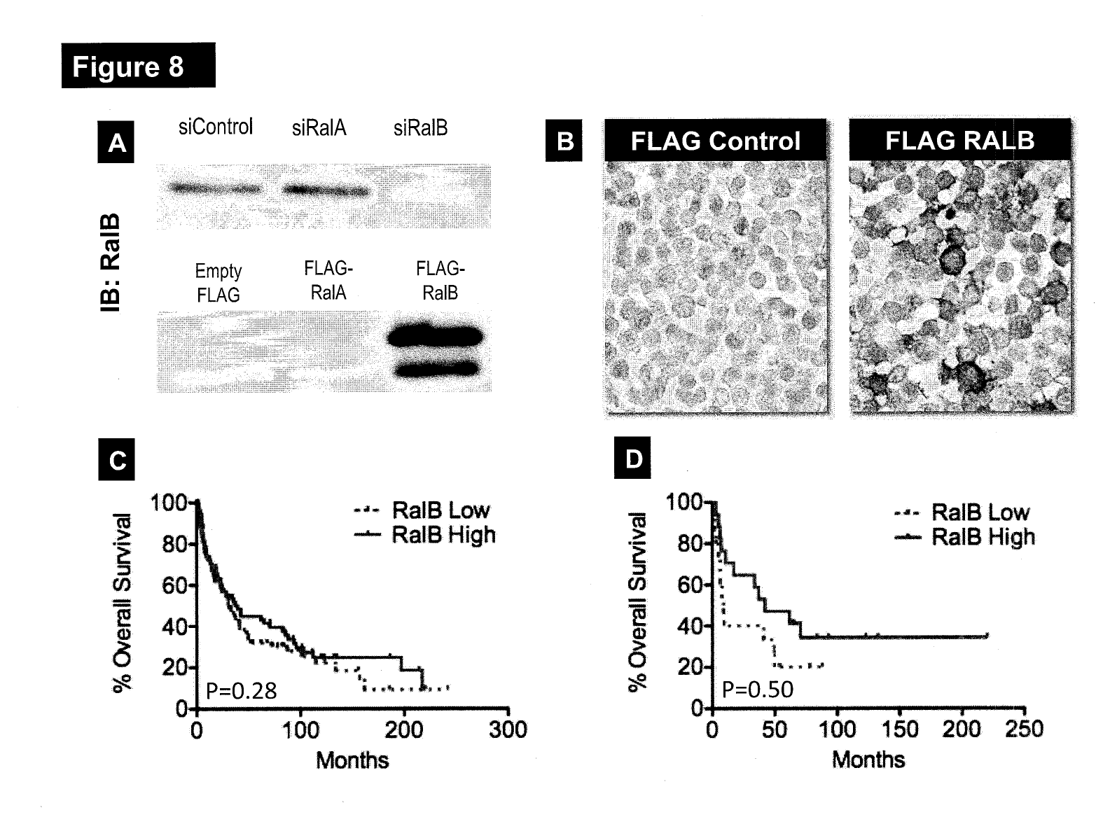

FIG. 9 or S3 shows Kaplan-Meier analyses and log rank test for trend P-value comparing overall survival over time for urothelial bladder cancer (N=104). Cases were stratified in three classes: RalA Low/RalB Low, RalA Low/RalB High & RalA High/RalB Low, or RalA High/RalB High. While the trend in survival was that of decreasing survival as a function of increased staining of both GTPases, this was not significant (log rank P=0.45) compared to that of RalA alone (P=0.16. Wilcoxon P=0.04, FIG. 1B).

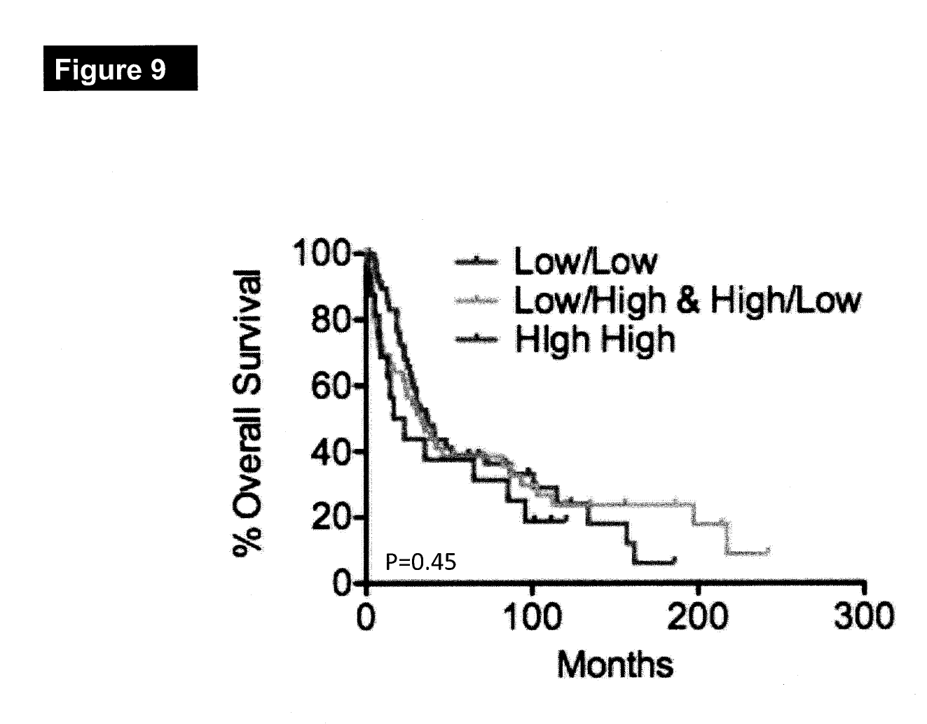

FIG. 10 or S4 shows Ral signature in two additional cohorts of bladder cancer cases. A. Using data from a cohort of bladder cancers (N=165) reported recently (Kim et al), significant difference was observed in distributions of Ral signature scores between non-muscle invasive (Ta/T1) and muscle invasive bladder cancers (T2+), Mann-Whitney U-test. B. Using data from a cohort of bladder cancers including nonmuscle invasive cases, non-muscle invasive cases showing subsequent progression, and muscle invasive cases, an overall significant difference was observed in distributions (Kruskal-Wallis) as well as, specifically, a significant difference between non-muscle invasive cases with and without subsequent progression (U-test).

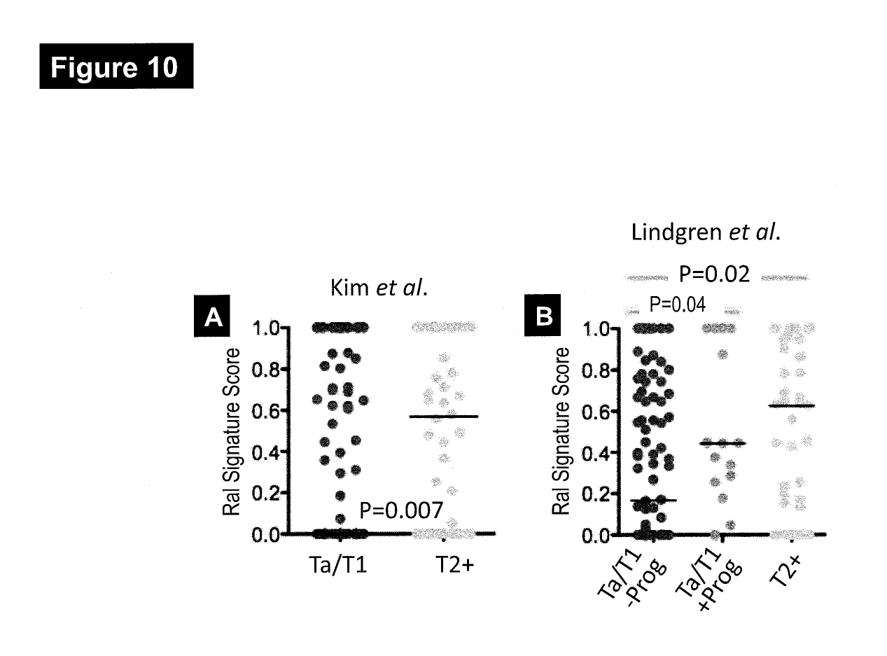

### DETAILED DESCRIPTION OF THE INVENTION

The present inventors have discovered polypeptides and polynucleotides that are differentially expressed in biological samples obtained from various cancer subjects. The levels and activities of these polypeptides and polynucleotides, along with clinical parameters can be used as biological markers indicative of the presence of cancer. The invention generally relates to the identification of a number of polypeptides and polynucleotides that are expressed in cancer patients and that are indicative of cancer parameters such as disease progression, metastasis and patient survival. Collectively, these polypeptides and polynucleotides constitute a gene expression signature of the Ral (Ras-like) GTPase protein, which was derived by identifying the genes regulated by Ral in several human tumor types. In various embodiments, the cancer may be bladder cancer, prostate cancer or squamous cell carcinoma.

According to one definition, a biological marker (“biomarker” or “marker”) is “a characteristic that is objectively measured and evaluated as an indicator of normal biologic processes, pathogenic processes, or pharmacological responses to therapeutic interventions.” NIH Biomarker Definitions Working Group (1998). Biomarkers can also include patterns or ensembles of characteristics indicative of particular biological processes (“panel of markers”). The biomarker measurement can increase or decrease to indicate a particular biological event or process. In addition, if a biomarker measurement typically changes in the absence of a particular biological process, a constant measurement can indicate occurrence of that process.

Marker measurements may be of the absolute values (e.g., the molar concentration of a molecule in a biological sample) or relative values (e.g., the relative concentration of two molecules in a biological sample). The quotient or product of two or more measurements also may be used as a marker. For example, some physicians rise the total blood cholesterol as a marker of the risk of developing coronary artery disease, while others use the ratio of total cholesterol to HDL cholesterol.

In the invention, the markers are primarily used for diagnostic and prognostic purposes. However they may also be used for therapeutic, drug screening and patient stratification purposes (e.g., to group patients into a number of “subsets” for evaluation), as well as other purposes described herein, including evaluation of the effectiveness of a cancer therapeutic.

The practice of the invention employs, unless otherwise indicated, conventional methods of analytical biochemistry, microbiology, molecular biology and recombinant DNA techniques generally known within the skill of the art. Such techniques are explained fully in the literature. (See, e.g., Sambrook et al. Molecular Cloning: A Laboratory Manual. 3rd, ed., Cold Spring Harbor Laboratory, Cold Spring Harbor Laboratory Press, Cold Spring Harbor, N.Y., 2000; DNA Cloning: A Practical Approach, Vol. 1 & 11 (Glover, ed.); Oligonucleotide Synthesis (Gait, ed., Current Edition); Nucleic Acid Hybridization (Flames & Higgins, eds., Current Edition); Transcription and Translation (Hames & Higgins, eds., Current Edition); CRC Handbook of Parvoviruses, Vol. I & II (Tijessen, ed.); Fundamental Virology, 2nd Edition, Vol. I & 11 (Fields and Knipe, eds.)).

The terminology used herein is for describing particular embodiments and is not intended to be limiting. As used herein, the singular forms “a,” “and” and “the” include plural referents unless the content and context clearly dictate otherwise. Thus, for example, a reference to “a marker” includes a combination of two or more such markers. Unless defined otherwise, all scientific and technical terms are to be understood as having the same meaning as commonly used in the art to which they pertain. For the purposes of the present invention, the following terms are defined below.

As used herein, the term “marker” includes polypeptide markers and polynucleotide markers. For clarity of disclosure, aspects of the invention will be described with respect to “polypeptide markers” and “polynucleotide markers.” However, statements made herein with respect to “polypeptide markers” are intended to apply to other polypeptides of the invention. Likewise, statements made herein with respect to “polynucleotide” markers are intended to apply to other polynucleotides of the invention, respectively. Thus, for example, a polynucleotide described as encoding a “polypeptide marker” is intended to include a polynucleotide that encodes: a polypeptide marker, a polypeptide that has substantial sequence identity to a polypeptide marker, modified polypeptide markers, fragments of a polypeptide marker, precursors of a polypeptide marker and successors of a polypeptide marker, and molecules that comprise a polypeptide marker, homologous polypeptide, a modified polypeptide marker or a fragment, precursor or successor of a polypeptide marker (e.g., a fusion protein).

As used herein, the term “polypeptide” refers to a polymer of amino acid residues that has at least 5 contiguous amino acid residues, e.g., 5, 6, 7, 8, 9, 10, 11 or 12 or more amino acids long, including each integer up to the full length of the polypeptide. A polypeptide may be composed of two or more polypeptide chains. A polypeptide includes a protein, a peptide, an oligopeptide, and an amino acid. A polypeptide can be linear or branched. A polypeptide can comprise modified amino acid residues, amino acid analogs or non-naturally occurring amino acid residues and can be interrupted by non-amino acid residues. Included within the definition are amino acid polymers that have been modified, whether naturally or by intervention, e.g., formation of a disulfide bond, glycosylation, lipidation, methylation, acetylation, phosphorylation, or by manipulation, such as conjugation with a labeling component. Also included are antibodies produced by a subject in response to overexpressed polypeptide markers.

As used herein, a “fragment” of a polypeptide refers to a single amino acid or a plurality of amino acid residues comprising an amino acid sequence that has at least 5 contiguous amino acid residues, at least 10 contiguous amino acid residues, at least 20 contiguous amino acid residues or at least 30 contiguous amino acid residues of a sequence of the polypeptide. As used herein, a “fragment” of polynucleotide refers to a single nucleic acid or to a polymer of nucleic acid residues comprising a nucleic acid sequence that has at least 15 contiguous nucleic acid residues, at least 30 contiguous nucleic acid residues, at least 60 contiguous nucleic acid residues, or at least 90% of a sequence of the polynucleotide. In some embodiment, the fragment is an antigenic fragment, and the size of the fragment will depend upon factors such as whether the epitope recognized by an antibody is a linear epitope or a conformational epitope. Thus, some antigenic fragments will consist of longer segments while others will consist of shorter segments, (e.g. 5, 6, 7, 8, 9, 10, 11 or 12 or more amino acids long, including each integer up to the full length of the polypeptide). Those skilled in the art are well versed in methods for selecting antigenic fragments of proteins.

In some embodiments, a polypeptide marker is a member of a biological pathway. As used herein, the term “precursor” or “successor” refers to molecules that precede or follow the polypeptide marker or polynucleotide marker in the biological pathway. Thus, once a polypeptide marker or polynucleotide marker is identified as a member of one or more biological pathways, the present invention can include additional precursor or successor members of the biological pathway. Such identification of biological pathways and their members is within the skill of one in the art.

As used herein, the term “polynucleotide” refers to a single nucleotide or a polymer of nucleic acid residues of any length. The polynucleotide may contain deoxyribonucleotides, ribonucleotides, and/or their analogs and may be double-stranded or single stranded. A polynucleotide can comprise modified nucleic acids (e.g., methylated), nucleic acid analogs or non-naturally occurring nucleic acids and can be interrupted by non-nucleic acid residues. For example a polynucleotide includes a gene, a gene fragment, cDNA, isolated DNA, mRNA, tRNA, rRNA, isolated RNA of any sequence, recombinant polynucleotides, primers, probes, plasmids, and vectors. Included within the definition are nucleic acid polymers that have been modified, whether naturally or by intervention.

As used herein, a component (e.g., a marker) is referred to as “differentially expressed” in one sample as compared to another sample when the method used for detecting the component provides a different level or activity when applied to the two samples. A component is referred to as “increased” in the first sample if the method for detecting the component indicates that the level or activity of the component is higher in the first sample than in the second sample (or if the component is detectable in the first sample but not in the second sample). Conversely, a component is referred to as “decreased” in the first sample if the method for detecting the component indicates that the level or activity of the component is lower in the first sample than in the second sample (or if the component is detectable in the second sample but not in the first sample). In particular, marker is referred to as “increased” or “decreased” in a sample (or set of samples) obtained from a cancer subject (or a subject who is suspected of having cancer, or is at risk of developing cancer) if the level or activity of the marker is higher or lower, respectively, compared to the level of the marker in a sample (or set of samples) obtained from a non-cancer subject, or a reference value or range.

The markers identified as being expressed in human cancer are of significant biologic interest and constitute a transcriptional signature of Ral proteins RalA and RalB that is associated with human tumors characteristics. As described herein, the status and clinical relevance of Ral was investigated in several human cancers by demonstrating ° immunohistochemistry of RalA and RalB and coupling that with evaluation of the transcriptional output of these proteins as a surrogate of Ral pathway activity. The data indicated that transcriptional signatures of Ral are associated with human tumor characteristics and patient outcomes, demonstrating systematically for the first time the clinical significance of Ral in human cancer.

As described in detail in Example 2, the transcriptional signature of Ral pathway status was developed based on profiling cells depleted of RalA or RalB. siRNA was used to deplete RalA or RalB from human bladder cancer cells and then the resultant transcriptional changes were profiled by microarray (Oxford et al 2007). Given the significant overlap between RalA and RalB-dependent transcriptional targets, a “core” signature of the transcriptional program common to both RalA and RalB was developed by choosing a union of 60 probesets regulated by RalA and RalB depletion in human bladder cancer cells (minimum 2 fold, >100 microarray expression units difference between closest replicates, Table S4). To this was applied the COXEN (co-expression extrapolation) principle (Lee et at 2007, Smith et at 2010) to define a subset of 39 probesets maintaining concordant expression in a published bladder cancer microarray cohort of patients treated by radical cystectomy (N=91) reported by Sanchez-Carbayo et al. (Sanchez-Carbayo et at 2006). These 39 probesets and corresponding genes are listed in Table S5.

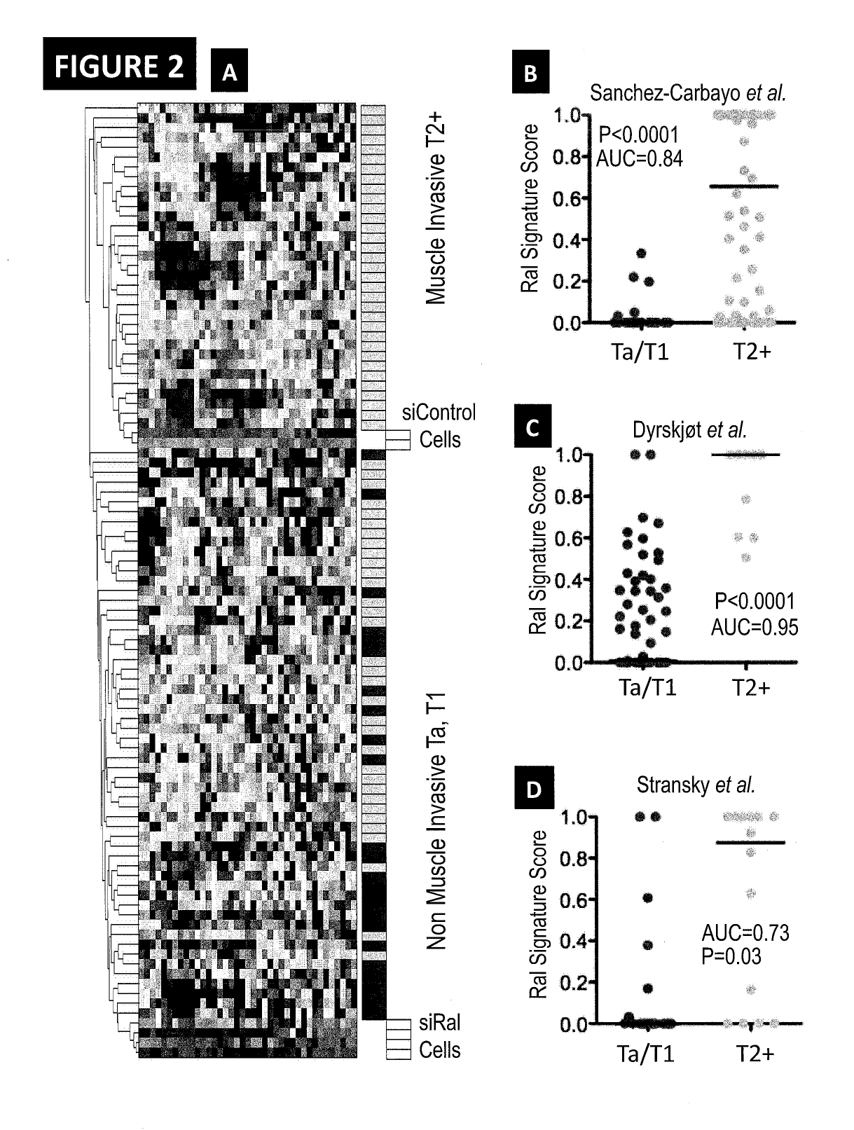

Using the 39 Ral signature probes of Table S5, the 91 tumors of the Sanchez-Carbayo cohort were clustered with control or Ral-depleted cells and it was round that non-muscle invasive (stage pTa, 071) tumors clustered with the Ral-depleted cells, while muscle invasive (stage T2+) tumors clustered with control treated cells (FIG. 2A, see Example 3). A significant difference in distributions of Ral signature scores between non-muscle invasive bladder cancers (NMIBC) and muscle invasive bladder cancers (MIBC), P<0.0001 (FIG. 2B), where NMIBCs had lower Ral signature scores and MIBCs had higher Ral signature scores. Finally, application of this signature to classify tumors of four additional independent cohorts of bladder tumors (Dyrskjot et al 2003, Kim et al 2010, Lindgren et al 2010, Stransky et al 2006) profiled on four different microarray platforms (total N=410) showed similar results (FIG. 2C-D, Figure S4A-B). Thus, it was found that Ral Signature is associated with invasive disease in human bladder cancer. Additionally, high Ral signature scores were found to be associated with metastatic and stem cell characteristics of bladder cancer cells, as well as with poor patient survival and disease progression in bladder cancer. See Examples 5 and 6.

In contrast, human squamous cell carcinoma cells were found to have a lower Ral Signature Score than normal mucosa. See Example 7. This is consistent with the recent reports suggesting that Ral may play a tumor suppressor role in squamous cell carcinoma (Sowalsky et al 2010, Sowalsky et al 2011).

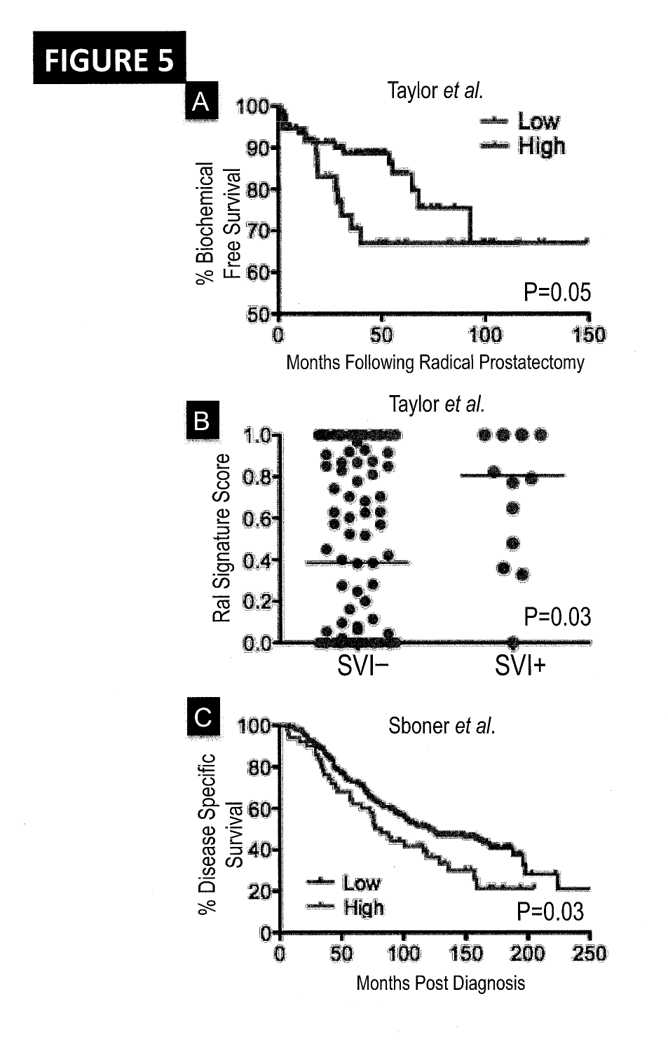

Ral signature was further investigated in human prostate cancer. See Example 8. Ral signature scores could risk stratify patients as a function of biochemical recurrence (P=0.05, FIG. 5A). Analogous to the association of high Ral signature score with invasion in bladder cancer, Ral signature scores were significantly higher in cases showing seminal vesicle invasion, a poor prognostic factor (P=0.028, FIG. 5B). In another cohort, Ral signature score was significantly correlated with Gleason score and could stratify the cases by disease specific survival (P=0.03, FIG. 5C). Additionally, Ral signature score distributions showed that higher scores of Ral are associated with androgen independent disease (FIG. 5A, P=0.005).

To our knowledge, the findings in this application provide the first evidence, sourced from tumor samples, supporting a role of Ral in mediating clinically meaningful phenotypes in human cancer. Findings regarding the novel Ral transcriptional signature of this invention closely parallel experimentally demonstrated roles of Ral in model systems.

The core signature of Ral-dependent transcription shared by RalA and RalB is a pervasive feature of muscle-invasive bladder cancer, and is consistent across a large number of cohorts from different institutions, geographical locations, and profiled on different microarray platforms. In the case of one cohort by Sanchez-Carbayo et al. where survival data were available, the signature was found to be associated with poor survival, consistent with the role of Ral in experimental metastasis (Wang et al 2010) as well as our observation herein that the Ral signature is associated with metastatic competence in experimental models (Overdevest et al 2011).

In prostate cancer, significant association of the Ral signature was found with androgen independence in two different cohorts. These findings implicate Ral in recurrence under androgen ablation therapy, a key driver of mortality in this disease.

In squamous cell carcinoma (SCC), where Ral was shown to act as a tumor suppressor in experimental systems in contrast to its role in other models (Sowalsky et al 2010), the Ral transcriptional signature score was lower in tumors compared to normal mucosa. This finding also speaks to the relative specificity of the Ral signature score. For example, if the score were simply a surrogate of a global phenotype such as transformation, one would not have expected to have observed lower signature scores in SCC compared to normal mucosa.

Thus, the findings of the present application provide a new tool, the Ral Signature score, that can be evaluated and compared to other prognostic tools in evaluating patients with cancers where Ral has been shown to have a driving role in model systems. Additionally, by demonstrating the clinical relevance of Ral in human tumors, the present work makes a strong case for investigation of strategies to interrupt Ral function.

The polynucleotide markers comprising the Ral signature set forth in Table S5 are also described by their HUGO identification symbol. The HUGO Gene Nomenclature Committee (HGNC) has assigned unique gene symbols and names to more than 32,000 human loci. genenames.org is a curated online repository of HGNC-approved gene nomenclature and associated resources including links to genomic, proteomic and phenotypic information, as well as dedicated gene family pages. All information associated with the publicly-available identifiers and accession numbers in any of the tables described herein, including the nucleic acid sequences of the associated genes, is incorporated herein by reference in its entirety. Given the name of the protein (also referred to herein as the “full protein”; indicated as “Protein”), other peptide fragments of such measured proteins may be obtained (by whatever means), and such other peptide fragments are included within the scope of the invention. The methods of the present invention may be used to evaluate fragments of the listed molecules as well as molecules that contain an entire listed molecule, or at least a significant portion thereof (e.g., measured unique epitope), and modified versions of the markers. Accordingly, such fragments, larger molecules and modified versions are included within the scope of the invention.

Homologs and alleles of the polypeptide markers of the invention can be identified by conventional techniques. As used herein, a homolog to a polypeptide is a polypeptide from a human or other animal that has a high degree of structural similarity to the identified polypeptides. Identification of human and other organism homologs of polypeptide markers identified herein will be familiar to those of skill in the art. In general, nucleic acid hybridization is a suitable method for identification of homologous sequences of another species (e.g., human, cow, sheep), which correspond to a known sequence. Standard nucleic acid hybridization procedures can be used to identify related nucleic acid sequences of selected percent identity. For example, one can construct a library of cDNAs reverse transcribed from the mRNA of a selected tissue (e.g., colon) and use the nucleic acids that encode polypeptides identified herein to screen the library for related nucleotide sequences. The screening preferably is performed using high-stringency conditions (described elsewhere herein) to identify those sequences that are closely related by sequence identity. Nucleic acids so identified can be translated into polypeptides and the polypeptides can be tested for activity.

Additionally, the present invention includes polypeptides or polynucleotides that have substantially similar sequence identity to the polypeptides or polynucleotides of the present invention. As used herein, two polypeptides or polynucleotides have “substantial sequence identity” when there is at least about 70% sequence identity, at least about 80% sequence identity, at least about 90% sequence identity, at least about 95% sequence identity, at least about 99% sequence identity, and preferably 100% sequence identity between their amino acid or nucleic acid sequences, or when polynucleotides encoding the polypeptides are capable of forming a stable duplex with each other under stringent hybridization conditions.

For example, conservative amino acid substitutions may be made in polypeptides to provide functionally equivalent variants of the foregoing polypeptides, i.e., the variants retain the functional capabilities of the polypeptides. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references that compile such methods. For example, upon determining that a peptide is a cancer-associated polypeptide, one can make conservative amino acid substitutions to the amino acid sequence of the peptide, and still have the polypeptide retain its specific antibody-binding characteristics. Additionally, one skilled in the art will realize that allelic variants and SNPs will give rise to substantially similar polypeptides and the same or substantially similar polypeptide fragments.

A number of comparison studies were performed to identify the polypeptide or polynucleotide markers listed using various groups of cancer and non-cancer patients. The table S5 lists markers that were found to be differentially expressed with statistical significance. Accordingly, it is believed that these biomarkers are indicators of cancer such as bladder cancer, prostate cancer and SCC

Where a polypeptide marker was found to be statistically significant in a plurality of studies, the data associated with the observations of highest statistical significance is presented. Accordingly, in one aspect, the invention provides polypeptide biomarkers of cancer. In one embodiment, the invention provides an isolated component listed in Table S5. In another embodiment, the invention provides a polypeptide or polynucleotide having substantial sequence identity with a component set forth in Table S5. In another embodiment, the invention provides a molecule that comprises a foregoing polypeptide or polynucleotide. As used herein, a compound is referred to as “isolated” when it has been separated from at least one component with which it is naturally associated. For example, a polypeptide can be considered isolated if it is separated from contaminants including metabolites, polynucleotides and other polypeptides. Isolated molecules can be either prepared synthetically or purified from their natural environment. Standard quantification methodologies known in the art can be employed to obtain and isolate the molecules of the invention.

Some variation is inherent in the measurements of the physical and chemical characteristics of the markers. The magnitude of the variation depends to some extent on the reproducibility of the separation means and the specificity and sensitivity of the detection means used to make the measurement. Preferably, the method and technique used to measure the markers is sensitive and reproducible.

Polypeptides or polynucleotides corresponding to the markers identified in Table S5 reflect a single polypeptide or polynucleotide appearing in a database for which the component was a match. In general, the polypeptide or polynucleotide is the largest polypeptide or polynucleotide found in the database. But such a selection is not meant to limit the polypeptide or polynucleotide to those corresponding to the markers disclosed in Table S5. Accordingly, in another embodiment, the invention provides a polypeptide or polynucleotide that is a fragment, precursor, successor or modified version of a marker described in Table S5. In another embodiment, the invention includes a molecule that comprises a foregoing fragment, precursor, successor or modified polypeptide or polynucleotide.

Another embodiment of the present invention relates to an assay system including a plurality of antibodies, or antigen binding fragments thereof, or aptamers for the detection of the expression of biomarkers differentially expressed in patients with cancer. The plurality of antibodies, or antigen binding fragments thereof, or aptamers consist of antibodies, or antigen binding fragments thereof, or aptamers that selectively bind to proteins differentially expressed in cancer patients, and that can be detected as protein products using antibodies or aptamers. In addition, the plurality of antibodies, or antigen binding fragments thereof, or aptamers comprise antibodies, or antigen binding fragments thereof, or aptamers that selectively bind to proteins or portions thereof (e.g., peptides) encoded by any of the genes from the tables provided herein.

Certain embodiments of the present invention utilize a plurality of biomarkers that have been identified herein as being differentially expressed in subjects with cancer. As used herein, the terms “patient,” “subject” and “a subject who has cancer” and “cancer subject” are intended to refer to subjects who have been diagnosed with cancer. The terms “non-subject” and “a subject who does not have cancer” are intended to refer to a subject who has not been diagnosed with cancer, or who is cancer-free as a result of surgery to remove the diseased tissue. A non-cancer subject may be healthy and have no other disease, or they may have a disease other than cancer.

The plurality of biomarkers within the above-limitation includes at least two or more biomarkers (e.g., at least 2, 3, 4, 5, 6, and so on, in whole integer increments, up to all of the possible biomarkers) identified by the present invention, and includes any combination of such biomarkers. Such biomarkers are selected from any of the markers listed in the Table S5 provided herein. In a preferred embodiment, the plurality of biomarkers used in the present invention includes all of the biomarkers listed in Table S5.

The polypeptide and polynucleotide markers of the invention are useful in methods for diagnosing cancer, determining the extent and/or severity of the disease, monitoring progression of the disease and/or response to therapy. Such methods can be performed in human and non-human subjects. The markers are also useful in methods for treating cancer and for evaluating the efficacy of treatment for the disease. Such methods can be performed in human and non-human subjects. The markers may also be used as pharmaceutical compositions or in kits. The markers may also be used to screen candidate compounds that modulate their expression. The markers may also be used to screen candidate drugs for treatment of cancer. Such screening methods can be performed in human and non-human subjects.

Polypeptide markers may be isolated by any suitable method known in the art. Markers can be purified from natural sources by standard methods known in the art (e.g., chromatography, centrifugation, differential solubility, immunoassay). In one embodiment, markers may be isolated from a biological sample using the methods disclosed herein. In another embodiment, polypeptide markers may be isolated from a sample by contacting the sample with substrate-bound antibodies or aptamers that specifically bind to the markers.

The present invention also includes polynucleotide markers related to the polypeptide markers of the present invention. In one aspect, the invention provides polynucleotides that encode the polypeptides of the invention. The polynucleotide may be genomic DNA, cDNA, or mRNA transcripts that encode the polypeptides of the invention. In one embodiment, the invention provides polynucleotides that encode a polypeptide described in Table S5, or a molecule that comprises such a polypeptide.

In another embodiment, the invention provides polynucleotides that encode a polypeptide having substantial sequence identity with a component set forth in Table S5, or a molecule that comprises such a polypeptide.

In another embodiment, the invention provides polynucleotides that encode a polypeptide that is a fragment, precursor, successor or modified version of a marker described in Table S5, or a molecule that comprises such polypeptide.

In another embodiment, the invention provides polynucleotides that have substantial sequence similarity to a polynucleotide that encodes a polypeptide that is a fragment, precursor, successor or modified version of a marker described in Table S5, or a molecule that comprises such polypeptide. Two polynucleotides have “substantial sequence identity” when there is at least about 70% sequence identity, at least about 80% sequence identity, at least about 90% sequence identity, at least about 95% sequence identity or at least 99% sequence identity between their amino acid sequences or when the polynucleotides are capable of forming a stable duplex with each other under stringent hybridization conditions. Such conditions are described elsewhere herein. As described above with respect to polypeptides, the invention includes polynucleotides that are allelic variants, the result of SNPs, or that in alternative codons to those present in the native materials as inherent in the degeneracy of the genetic code.

In some embodiments, the polynucleotides described may be used as surrogate markers of the cancer. Thus, for example, if the level of a polypeptide marker is increased in bladder cancer-patients, an increase in the mRNA that encodes the polypeptide marker may be interrogated rather than the polypeptide marker (e.g., to diagnose bladder cancer in a subject).

Polynucleotide markers may be isolated by any suitable method known in the art. Native polynucleotide markers may be purified from natural sources by standard methods known in the art (e.g., chromatography, centrifugation, differential solubility, immunoassay). In one embodiment, a polynucleotide marker may be isolated from a mixture by contacting the mixture with substrate bound probes that are complementary to the polynucleotide marker under hybridization conditions.

Alternatively, polynucleotide markers may be synthesized by any suitable chemical or recombinant method known in the art. In one embodiment, for example, the makers can be synthesized using the methods and techniques of organic chemistry. In another embodiment, a polynucleotide marker can be produced by polymerase chain reaction (PCR).

The present invention also encompasses molecules which specifically bind the polypeptide or polynucleotide markers of the present invention. In one aspect, the invention provides molecules that specifically bind to a polypeptide marker or a polynucleotide marker. As used herein, the term “specifically binding,” refers to the interaction between binding pairs (e.g., an antibody and an antigen or aptamer and its target). In some embodiments, the interaction has an affinity constant of at most 10⁻⁶ moles/liter, at most 10⁻⁷ moles/liter, or at most 10⁻⁸ moles/liter. In other embodiments, the phrase “specifically binds” refers to the specific binding of one protein to another (e.g., an antibody, fragment thereof, or binding partner to an antigen), wherein the level of binding, as measured by any standard assay (e.g., an immunoassay), is statistically significantly higher than the background control for the assay. For example, when performing an immunoassay, controls typically include a reaction well/tube that contain antibody or antigen binding fragment alone (i.e., in the absence of antigen), wherein an amount of reactivity (e.g., non-specific binding to the well) by the antibody or antigen binding fragment thereof in the absence of the antigen is considered to be background. Binding can be measured using a variety of methods standard in the art including enzyme immunoassays (e.g., ELISA), immunoblot assays, etc.).

The binding molecules include antibodies, aptamers and antibody fragments. As used herein, the term “antibody” refers to an immunoglobulin molecule capable of binding an epitope present on an antigen. The term is intended to encompasses not only intact immunoglobulin molecules such as monoclonal and polyclonal antibodies, but also bi-specific antibodies, humanized antibodies, chimeric antibodies, anti-idiopathic (anti-ID) antibodies, single-chain antibodies, Fab fragments, F(ab′) fragments, fusion proteins and any modifications of the foregoing that comprise an antigen recognition site of the required specificity. As used herein, an aptamer is a non-naturally occurring nucleic acid having a desirable action on a target. A desirable action includes, but is not limited to, binding of the target, catalytically changing the target, reacting with the target in a way which modifies/alters the target or the functional activity of the target, covalently attaching to the target as in a suicide inhibitor, facilitating the reaction between the target and another molecule. in the preferred embodiment, the action is specific binding affinity for a target molecule, such target molecule being a three dimensional chemical structure other than a polynucleotide that binds to the nucleic acid ligand through a mechanism which predominantly depends on Watson/Crick base pairing or triple helix binding, wherein the nucleic acid ligand is not a nucleic acid having the known physiological function of being bound by the target molecule.

In one aspect, the invention provides antibodies or aptamers that specifically bind to a component listed in Table S5, or to a molecule that comprises a foregoing component (e.g., a protein comprising a polypeptide identified in a table of the invention).

In another embodiment, the invention provides antibodies or aptamers that specifically bind to a polypeptide having substantial sequence identity with a component set forth in Table S5, or to a molecule that comprises a foregoing polypeptide.

In another embodiment, the invention provides antibodies or aptamers that specifically bind to a component that is a fragment, modification, precursor or successor of a marker described in Table S5, or to a molecule that comprises a foregoing component.

In another embodiment, the invention provides antibodies or aptamers that specifically bind to a polypeptide marker or a polynucleotide marker that is structurally different from a component specifically identified in Table S5 but has the same (or nearly the same) function or properties, or to a molecule that comprises a foregoing component.

Another embodiment of the present invention relates to a plurality of aptamers, antibodies, or antigen binding fragments thereof, for the detection of the expression of biomarkers differentially expressed in patients with cancer. The plurality of aptamers, antibodies, or antigen binding fragments thereof, consists of antibodies, or antigen binding fragments thereof, that selectively bind to proteins differentially expressed in patients with cancer, and that can be detected as protein products using antibodies. In addition, the plurality of aptamers, antibodies, or antigen binding fragments thereof; comprises antibodies, or antigen binding fragments thereof, that selectively bind to proteins or portions thereof (peptides) encoded by any of the genes from the tables provided herein.

According to the present invention, a plurality of aptamers, antibodies, or antigen binding fragments thereof, refers to at least 2, and more preferably at least 3, and more preferably at least 4, and more preferably at least 5, and more preferably at least 6, and more preferably at least 7, and more preferably at least 8, and more preferably at least 9, and more preferably at least 10, and so on, in increments of one, up to any suitable number of antibodies, or antigen binding fragments thereof, including, in a preferred embodiment, antibodies representing all of the biomarkers described herein, or antigen binding fragments thereof.

Certain antibodies that specifically bind polypeptide markers polynucleotide markers of the invention already may be known and/or available for purchase from commercial sources. In any event, the antibodies of the invention may be prepared by any suitable means known in the art. For example, antibodies may be prepared by immunizing an animal host with a marker or an immunogenic fragment thereof (conjugated to a carrier, if necessary). Adjuvants (e.g., Freund's adjuvant) optionally may be used to increase the immunological response. Sera containing polyclonal antibodies with high affinity for the antigenic determinant can then be isolated from the immunized animal and purified.

Alternatively, antibody-producing tissue from the immunized host can be harvested and a cellular homogenate prepared from the organ can be fused to cultured cancer cells. Hybrid cells which produce monoclonal antibodies specific for a marker can be selected. Alternatively, the antibodies of the invention can be produced by chemical synthesis or by recombinant expression. For example, a polynucleotide that encodes the antibody can be used to construct an expression vector for the production of the antibody. The antibodies of the present invention can also be generated using various phage display methods known in the art.

Antibodies or aptamers that specifically bind markers of the invention can be used, for example, in methods for detecting components listed in Table S5 using methods and techniques well-known in the art. In some embodiments, for example, the antibodies are conjugated to a detection molecule or moiety (e.g., a dye, and enzyme) and can be used in ELISA or sandwich assays to detect markers of the invention.

In another embodiment, antibodies or aptamers against a polypeptide marker or polynucleotide marker of the invention can be used to assay a tissue sample (e.g., a thin cortical slice) for the marker. The antibodies or aptamers can specifically bind to the marker, if any, present in the tissue sections and allow the localization of the marker in the tissue. Similarly, antibodies or aptamers labeled with a radioisotope may be used for in vivo imaging or treatment applications.

Another aspect of the invention provides compositions comprising a polypeptide or polynucleotide marker of the invention, a binding molecule that is specific for a polypeptide or polynucleotide marker (e.g., an antibody or an aptamer), an inhibitor of a polypeptide or polynucleotide marker, or other molecule that can increase or decrease the level or activity of a polypeptide marker or polynucleotide marker. Such compositions may be pharmaceutical compositions formulated for use as a therapeutic.

Alternatively, the invention provides a composition that comprises a component that is a fragment, modification, precursor or successor of a marker described in Table S5, or to a molecule that comprises a foregoing component.

In another embodiment, the invention provides a composition that comprises a polynucleotide that binds to a polypeptide or a molecule that comprises a foregoing polynucleotide.

In another embodiment, the invention provides a composition that comprises an antibody or aptamer that specifically binds to a polypeptide or a molecule that comprises a foregoing antibody or aptamer.

The present invention also provides methods of detecting the biomarkers of the present invention. The practice of the present invention employs, unless otherwise indicated, conventional methods of analytical biochemistry, microbiology, molecular biology and recombinant DNA techniques within the skill of the art. Such techniques are explained fully in the literature. (See, e.g., Sambrook, J. et al. Molecular Cloning: A Laboratory Manual. 3rd, ed., Cold Spring Harbor Laboratory, Cold Spring Harbor Laboratory Press, Cold Spring Harbor, N.Y., 2000; DNA Cloning: A Practical Approach, Vol. I & II (D. Glover, ed.); Oligonucleotide Synthesis (N. Gait, ed., Current Edition); Nucleic Acid Hybridization (B. Hames & S. Higgins, eds., Current Edition); Transcription and Translation (B. Flames & S. Higgins, eds., Current Edition); CRC Handbook of Parvoviruses, Vol. 1 & II (P. Tijessen, ed.); Fundamental Virology, 2nd Edition, Vol. I & 11(13. N. Fields and D. M. Knipe, eds.)).

The markers of the invention may be detected by any method known to those of skill in the art, including without limitation LC-MS, GC-MS, immunoassays, hybridization and enzyme assays. The detection may be quantitative or qualitative. A wide variety of conventional techniques are available, including mass spectrometry, chromatographic separations, 2-D gel separations, binding assays (e.g., immunoassays), competitive inhibition assays, and so on. Any effective method in the art for measuring the presence/absence, level or activity of a marker is included in the invention. It is within the ability of one of ordinary skill in the art to determine which method would be most appropriate for measuring a specific marker. Thus, for example, an ELISA assay may be best suited for use in a physician's office while a measurement requiring more sophisticated instrumentation may be best suited for use in a clinical laboratory. Regardless of the method selected, it is important that the measurements be reproducible.

The markers of the invention can be measured by mass spectrometry, which allows direct measurements of analytes with high sensitivity and reproducibility. A number of mass spectrometric methods are available. As will be appreciated by one of skill in the art, many separation technologies may be used in connection with mass spectrometry. For example, a wide selection of separation columns is commercially available. In addition, separations may be performed using custom chromatographic surfaces (e.g., a bead on which a marker specific reagent has been immobilized). Molecules retained on the media subsequently may be eluted for analysis by mass spectrometry.

For protein markers, quantification can be based on derivatization in combination with isotopic labeling, referred to as isotope coded affinity tags (“ICAT”). In this and other related methods, a specific amino acid in two samples is differentially and isotopically labeled and subsequently separated from peptide background by solid phase capture, wash and release. The intensities of the molecules from the two sources with different isotopic labels can then be accurately quantified with respect to one another. Quantification can also be based on the isotope dilution method by spiking in an isotopically labeled peptide or protein analogous to those being measured. Furthermore, quantification can also be determined without isotopic standards using the direct intensity of the analyte comparing with another measurement of a standard in a similar matrix.

In addition, one- and two-dimensional gels have been used to separate proteins and quantify gels spots by silver staining, fluorescence or radioactive labeling. These differently stained spots have been detected using mass spectrometry, and identified by tandem mass spectrometry techniques.

In one embodiment, the markers are measured using mass spectrometry in connection with a separation technology, such as liquid chromatography-mass spectrometry or gas chromatography-mass spectrometry. In particular, coupling reverse-phase liquid chromatography to high resolution, high mass accuracy ESI time-of-flight (TOF) mass spectroscopy allows spectral intensity measurement of a large number of biomolecules from a relatively small amount of any complex biological material. Analyzing a sample in this manner allows the marker (characterized by a specific RT and m/z) to be determined and quantified.

As will be appreciated by one of skill in the art, many other separation technologies may be used in connection with mass spectrometry. For example, a wide selection of separation columns is commercially available. In addition, separations may be performed using custom chromatographic surfaces (e.g., a bead on which a marker specific reagent has been immobilized). Molecules retained on the media subsequently may be eluted for analysis by mass spectrometry.

Analysis by liquid chromatography-mass spectrometry produces a mass intensity spectrum, the peaks of which represent various components of the sample, each component having a characteristic mass-to-charge ratio (m/z) and retention time (RT). The presence of a peak with the m/z and RT of a marker indicates that the marker is present. The peak representing a marker may be compared to a corresponding peak from another spectrum (e.g., from a control sample) to obtain a relative measurement. Any normalization technique in the art (e.g., an internal standard) may be used when a quantitative measurement is desired. “Deconvoluting” software is available to separate overlapping peaks. The retention time depends to some degree on the conditions employed in performing the liquid chromatography separation. The preferred conditions, those used to obtain the retention times that appear in the Tables, are set forth in the Example. The mass spectrometer preferably provides high mass accuracy and high mass resolution. The mass accuracy of a well-calibrated Micromass TOF instrument, for example, is reported to be approximately 5 mDa, with resolution m/Δm exceeding 5000.

In other preferred embodiments, the level of the markers may be determined using a standard immunoassay, such as sandwiched ELISA using matched antibody pairs and chemiluminescent detection. Commercially available or custom monoclonal or polyclonal antibodies are typically used. However, the assay can be adapted for use with other reagents that specifically bind to the marker. Standard protocols and data analysis are used to determine the marker concentrations from the assay data.

A number of the assays discussed above employ a reagent that specifically binds to the marker. Any molecule that is capable of specifically binding to a marker is included within the invention. In some embodiments, the binding molecules are antibodies or antibody fragments. In other embodiments; the binding molecules are non-antibody species, such as aptamers. Thus, for example, the binding molecule may be an enzyme for which the marker is a substrate. The binding molecules may recognize any epitope of the targeted markers.

As described above, the binding molecules may be identified and produced by any method accepted in the art. Methods for identifying and producing antibodies and antibody fragments specific for an analyte are well known. Examples of other methods used to identify the binding molecules include binding assays with random peptide libraries (e.g., phage display) and design methods based on an analysis of the structure of the marker.

The markers of the invention also may be detected or measured using a number of chemical derivatization or reaction techniques known in the art. Reagents for use in such techniques are known in the art, and are commercially available for certain classes of target molecules.

Finally, the chromatographic separation techniques described above also may be coupled to an analytical technique other than mass spectrometry such as fluorescence detection of tagged molecules, NMR, capillary UV, evaporative light scattering or electrochemical detection.

Measurement of the relative amount of an RNA or protein marker of the invention may be by any method known in the art (see, e.g., Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989; and Current Protocols in Molecular Biology, eds. Ausubel et al. John Wiley & Sons: 1992). Typical methodologies for RNA detection include RNA extraction from a cell or tissue sample, followed by hybridization of a labeled probe (e.g., a complementary polynucleotide) specific for the target RNA to the extracted RNA, and detection of the probe (e.g., Northern blotting). Typical methodologies for protein detection include protein extraction from a cell or tissue sample, followed by hybridization of a labeled probe (e.g., an antibody) specific for the target protein to the protein sample, and detection of the probe. The label group can be a radioisotope, a fluorescent compound, an enzyme, or an enzyme co-factor. Detection of specific protein and polynucleotides may also be assessed by gel electrophoresis, column chromatography, direct sequencing, or quantitative PCR (in the case of polynucleotides) among many other techniques well known to those skilled in the art.

Detection of the presence or number of copies of all or a part of a marker gene of the invention may be performed using any method known in the art. Typically, it is convenient to assess the presence and/or quantity of a DNA or cDNA by Southern analysis, in which total DNA from a cell or tissue sample is extracted, is hybridized with a labeled probe (e.g., a complementary DNA molecule), and the probe is detected. The label group can be a radioisotope, a fluorescent compound, an enzyme, or an enzyme co-factor. Other useful methods of DNA detection and/or quantification include direct sequencing, gel electrophoresis, column chromatography, and quantitative PCR, as is known by one skilled in the art.

Polynucleotide similarity can be evaluated by hybridization between single stranded nucleic acids with complementary or partially complementary sequences. Such experiments are well known in the art. High stringency hybridization and washing conditions, as referred to herein, refer to conditions which permit isolation of nucleic acid molecules having at least about 80% nucleic acid sequence identity with the nucleic acid molecule being used to probe in the hybridization reaction (i.e., conditions permitting about 20% or less mismatch of nucleotides). Very high stringency hybridization and washing conditions, as referred to herein, refer to conditions which permit isolation of nucleic acid molecules having at least about 90% nucleic acid sequence identity with the nucleic acid molecule being used to probe in the hybridization reaction (i.e., conditions permitting about 10% or less mismatch of nucleotides). As discussed above, one of skill in the art can use the formulae in Meinkoth et al., ibid. to calculate the appropriate hybridization and wash conditions to achieve these particular levels of nucleotide mismatch. Such conditions will vary, depending on whether DNA:RNA or DNA:DNA hybrids are being formed. Calculated melting temperatures for DNA:DNA hybrids are 10° C. less than for DNA:RNA hybrids. In particular embodiments, stringent hybridization conditions for DNA:DNA hybrids include hybridization at an ionic strength of 6×SSC (0.9 M Na⁺) at a temperature of between about 20° C. and about 35° C. (lower stringency), more preferably, between about 28° C. and about 40° C. (more stringent), and even more preferably, between about 35° C. and about 45° C. (even more stringent), with appropriate wash conditions. In particular embodiments, stringent hybridization conditions for DNA:RNA hybrids include hybridization at an ionic strength of 6×SSC (0.9 M Na⁺) at a temperature of between about 30° C. and about 45° C., more preferably, between about 38° C. and about 50° C., and even more preferably, between about 45° C. and about 55° C., with similarly stringent wash conditions. These values are based on calculations of a melting temperature for molecules larger than about 100 nucleotides, 0% formamide and a G+C content of about 40%. Alternatively, Tₘ can be calculated empirically as set forth in Sambrook et al., supra, pages 9.31 to 9.62. In general, the wash conditions should be as stringent as possible, and should be appropriate for the chosen hybridization conditions. For example, hybridization conditions can include a combination of salt and temperature conditions that are approximately 20-25° C. below the calculated Tₘ of a particular hybrid, and wash conditions typically include a combination of salt and temperature conditions that are approximately 12-20° C. below the calculated Tₘ of the particular hybrid. One example of hybridization conditions suitable for use with DNA:DNA hybrids includes a 2-24 hour hybridization in 6×SSC (50% formamide) at about 42° C., followed by washing steps that include one or more washes at room temperature in about 2×SSC, followed by additional washes at higher temperatures and lower ionic strength (e.g., at least one wash as about 37° C. in about 0.1×-0.5×SSC, followed by at least one wash at about 68° C. in about 0.1×-0.5×SSC). Other hybridization conditions, and for example, those most useful with nucleic acid arrays, will be known to those of skill in the art.

The present invention also includes methods of diagnosing cancer and related methods. In general, it is expected that the biomarkers described herein will be measured in combination with other signs, symptoms and clinical tests of bladder, prostate or SCC cancer, such as MRI or ultrasound abnormalities, or other cancer biomarkers reported in the literature. Likewise, more than one of the biomarkers of the present invention may be measured in combination. Measurement of the biomarkers of the invention along with any other markers known in the art, including those not specifically listed herein, falls within the scope of the present invention. Markers appropriate for this embodiment include those that have been identified as increased or decreased in samples obtained from cancer samples compared with samples from non-cancer samples (e.g., markers described in Table S5, as well as antibodies produced by a patient in response to an increased level of a polypeptide marker. Other markers appropriate for this embodiment include fragments, precursors, successors and modified versions of such markers, polypeptides having substantial sequence identity to such markers, components having an m/z value and RT value of about the values set forth for the markers described in Table S5, and molecules comprise one of the foregoing. Other appropriate markers for this embodiment will be apparent to one of skill in the art in light of the disclosure herein.

In one embodiment, the present invention provides a method for determining whether a subject has bladder, prostate or SCC cancer. In another aspect, the invention provides methods for diagnosing cancer in a subject. These methods comprise obtaining a biological sample from a subject suspected of having the cancer, or at risk for developing the cancer, detecting the level or activity of one or more biomarkers in the sample, and comparing the result to the level or activity of the marker(s) in a sample obtained from a non-cancer subject, or to a reference range or value. As used herein, the term “biological sample” includes a sample from any body fluid or tissue (e.g., serum, plasma, blood, cerebrospinal fluid, urine, saliva, cancer tissue). Typically, the standard biomarker level or reference range is obtained by measuring the same marker or markers in a set of normal controls. Measurement of the standard biomarker level or reference range need not be made contemporaneously; it may be a historical measurement. Preferably the normal control is matched to the patient with respect to some attribute(s) (e.g., age). Depending upon the difference between the measured and standard level or reference range, the patient can be diagnosed as having cancer or as not having cancer. In some embodiments, cancer is diagnosed in the patient if the expression level of the biomarker or biomarkers in the patient sample is statistically more similar to the expression level of the biomarker or biomarkers that has been associated with cancer than the expression level of the biomarker or biomarkers that has been associated with the normal controls.

What is presently referred to as bladder or prostate cancer may turn out to be a number of related, but distinguishable conditions. Classifications may be made, and these types may be further distinguished into subtypes. Indeed, by providing a method for subsetting patients based on biomarker measurement level, the compositions and methods of the present invention may be used to uncover and define various forms of the disease.

The methods of the present invention may be used to make the diagnosis of bladder, prostate or SCC cancer, independently from other information such as the patient's symptoms or the results of other clinical or paraclinical tests. However, the methods of the present invention may be used in conjunction with such other data points.

Because a diagnosis is rarely based exclusively on the results of a single test, the method may be used to determine whether a subject is more likely than not to have cancer, or is more likely to have cancer than to have another disease, based on the difference between the measured and standard level or reference range of the biomarker. Thus, for example, a patient with a putative diagnosis of cancer may be diagnosed as being “more likely” or “less likely” to have cancer in light of the information provided by a method of the present invention. If a plurality of biomarkers are measured, at least One and up to all of the measured biomarkers must differ, in the appropriate direction, for the subject to be diagnosed as having (or being more likely to have) cancer. In some embodiments, such difference is statistically significant.

The biological sample may be of any tissue or fluid, including a serum or tissue sample, but other biological fluids or tissue may be used. Possible biological samples include, but are not limited to, blood, plasma, urine, saliva, and cancer tissue: In some embodiments, the level of a marker may be compared to the level of another marker or some other component in a different tissue, fluid or biological “compartment.” Thus, a differential comparison may be made of a marker in tissue and serum. It is also within the scope of the invention to compare the level of a marker with the level of another marker or some other component within the same compartment.

As will be apparent to those of ordinary skill in the art, the above description is not limited to making an initial diagnosis of cancer, but also is applicable to confirming a provisional diagnosis of cancer or “ruling out” such a diagnosis. Furthermore, an increased or decreased level or activity of the marker(s) in a sample obtained from a subject suspected of having cancer, or at risk for developing cancer, is indicative that the subject has or is at risk for developing cancer.

The invention also provides a method for determining a subject's risk of developing cancer, the method comprising obtaining a biological sample from a subject, detecting the level or activity of a marker in the sample, and comparing the result to the level or activity of the marker in a sample obtained from a non-cancer subject, or to a reference range or value wherein an increase or decrease of the marker is correlated with the risk of developing cancer.

The invention also provides methods for determining the stage or severity of cancer, the method comprising obtaining a biological sample from a subject, detecting the level or activity of a marker in the sample, and comparing the result to the level or activity of the marker in a sample obtained from a non-cancer subject, or to a reference range or value wherein an increase or decrease of the marker is correlated with the stage or severity of the disease.

In another aspect, the invention provides methods for monitoring the progression of the disease in a subject who has cancer, the method comprising obtaining a first biological sample from a subject, detecting the level or activity of a marker in the sample, and comparing the result to the level or activity of the marker in a second sample obtained from the subject at a later time, or to a reference range or value wherein an increase or decrease of the marker is correlated with progression of the disease.

Cancer prognosis generally refers to a forecast or prediction of the probable course or outcome of the cancer. As used herein, cancer prognosis includes the forecast or prediction of any one or more of the following: duration of survival of a patient susceptible to or diagnosed with a cancer, duration of recurrence-free survival, duration of progression free survival of a patient susceptible to or diagnosed with a cancer, response rate in a group of patients susceptible to or diagnosed with a cancer, duration of response in a patient or a group of patients susceptible to or diagnosed with a cancer, and/or likelihood of metastasis in a patient susceptible to or diagnosed with a cancer. Prognostic for cancer means providing a forecast or prediction of the probable course or outcome of the cancer. In some embodiments, prognostic for cancer comprises providing the forecast or prediction of (prognostic for) any one or more of the following: duration of survival of a patient susceptible to or diagnosed with a cancer, duration of recurrence-free survival, duration of progression free survival of a patient susceptible to or diagnosed with a cancer, response rate in a group of patients susceptible to or diagnosed with a cancer, duration of response in a patient or a group of patients susceptible to or diagnosed with a cancer, and/or likelihood of metastasis in a patient susceptible to or diagnosed with a cancer.

The marker expression measurement values for the markers listed in Table S5 are differentially expressed in cancer samples. For markers that are increased or upregulated, a significant difference in the elevation of the measured value of one or more of the markers indicates that the patient has (or is more likely to have, or is at risk of having, or is at risk of developing, and so forth) cancer. For markers that are decreased or downregulated, a significant difference in the depression of the measured value of one or more of the markers indicates that the patient has (or is more likely to have, or is at risk of having, or is at risk of developing, and so forth) cancer. If only one biomarker is measured, then that value must change (either increase or decrease) to indicate cancer. If more than one biomarker is measured, then a diagnosis of cancer can be indicated by a change in only one biomarker, all biomarkers, or any number in between. In some embodiments, multiple markers are measured, and a diagnosis of cancer is indicated by changes in multiple markers. For example, a panel of markers may include markers that are increased in level or activity in cancer subject samples as compared to non-cancer subject samples, markers that are decreased in level or activity in cancer subject samples as compared to non-cancer subject samples, or a combination thereof. Measurements can be of (i) a biomarker of the present invention, (ii) a biomarker of the present invention and another factor known to be associated with cancer (e.g., alpha-fetoprotein (APP), abdominal ultrasound, helical CT scan and/or triple phase CT scan); (iii) a plurality of biomarkers of the present invention, (iv) a plurality of biomarkers comprising at least one biomarker of the present invention and at least one biomarker reported in the literature; or (v) any combination of the foregoing. Furthermore, the amount of change in a biomarker level may be an indication of the relative likelihood of the presence of the disease.

The marker(s) may be detected in any biological sample obtained from the subject, by any suitable method known in the art (e.g., immunoassays, hybridization assay) see supra. In some embodiments, the marker(s) are detected in a tumor sample obtained from the patient by surgical procedure(s).

In an alternative embodiment of the invention, a method is provided for monitoring a cancer patient over time to determine whether the disease is progressing. The specific techniques used in implementing this embodiment are similar to those used in the embodiments described above. The method is performed by obtaining a biological sample, such as serum or tissue, from the subject at a certain time (t₁); measuring the level of at least one of the biomarkers in the biological sample; and comparing the measured level with the level measured with respect to a biological sample obtained from the subject at an earlier time (t₀). Depending upon the difference between the measured levels, it can be seen whether the marker level has increased, decreased, or remained constant over the interval (t₁−t₀). A further deviation of a marker in the direction indicating cancer, or the measurement of additional increased or decreased cancer markers, would suggest a progression of the disease during the interval. Subsequent sample acquisitions and measurements can be performed as many times as desired over a range of times t₂ to tₙ.

The ability to monitor a patient by making serial marker level determinations would represent a valuable clinical tool. Rather than the limited “snapshot” provided by a single test, such monitoring would reveal trends in marker levels over time. In addition to indicating a progression of the disease, tracking the marker levels in a patient could be used to predict exacerbations or indicate the clinical course of the disease. For example, as will be apparent to one of skill in the art, the biomarkers of the present invention could be further investigated to distinguish between any or all of the known forms of cancer or any later described types or subtypes of the disease. In addition, the sensitivity and specificity of any method of the present invention could be further investigated with respect to distinguishing cancer from other diseases or to predict relapse or remission.

In an analogous manner, administration of a chemotherapeutic drug or drug combination can be evaluated or re-evaluated in light of the assay results of the present invention. For example, the drug(s) can be administered differently to different subject populations, and measurements corresponding to administration analyzed to determine if the differences in the inventive biomarker signature before and after drug administration are significant. Results from the different drug regiments can also be compared with each other directly. Alternatively, the assay results may indicate the desirability of one drug regimen over another, or indicate that a specific drug regimen should or should not be administered to a cancer patient. In one embodiment, the finding of elevated levels of the markers of the present invention in a cancer patient is indicative of a good prognosis for response to treatment with chemotherapeutic agents. In another embodiment, the absence of elevated levels of the markers of the present invention in a cancer patient is indicative of a poor prognosis for response to treatment.

In another aspect, the invention provides methods for screening candidate compounds for use as therapeutic compounds. In one embodiment, the method comprises screening candidate compounds for those that provide clinical progress following administration to a cancer patient from which a tumor sample has been shown to have elevated levels of the markers of the present invention.

In an analogous manner, the markers of the present invention can be used to assess the efficacy of a therapeutic intervention in a subject. The same approach described above would be used, except a suitable treatment would be started, or an ongoing treatment would be changed, before the second measurement (i.e., after t₀ and before t₁). The treatment can be any therapeutic intervention, such as drug administration, dietary restriction or surgery, and can follow any suitable schedule over any time period as appropriate for the intervention. The measurements before and after could then be compared to determine whether or not the treatment had an effect effective. As will be appreciated by one of skill in the art, the determination may be confounded by other superimposed processes (e.g., an exacerbation of the disease during the same period).

In a further embodiment, the markers may be used to screen candidate drugs, For example, in a clinical trial, to determine whether a candidate drug is effective in treating cancer. At time tₒ, a biological sample is obtained from each subject in population of subjects diagnosed with cancer. Next, assays are performed on each subject's sample to measure levels of a biological marker. In some embodiments, only a single marker is monitored, while in other embodiments, a combination of markers, up to the total number of factors, is monitored. Next, a predetermined dose of a candidate drug is administered to a portion or sub-population of the same subject population. Drug administration can follow any suitable schedule over any time period. In some cases, varying closes are administered to different subjects within the sub-population, or the drug is administered by different routes. At time t₁, after drug administration, a biological sample is acquired from the sub-population and the same assays are performed on the biological samples as were previously performed to obtain measurement values. As before, subsequent sample acquisitions and measurements can be performed as many times as desired over a range of times t₂ to tₙ. In such a study, a different sub-population of the subject population serves as a control group, to which a placebo is administered. The same procedure is then followed for the control group: obtaining the biological sample, processing the sample, and measuring the biological markers to obtain a measurement chart.

Specific doses and delivery routes can also be examined. The method is performed by administering the candidate drug at specified dose or delivery routes to subjects with cancer; obtaining biological samples, such as serum or tissue, from the subjects; measuring the level of at least one of the biomarkers in each of the biological samples; and, comparing the measured level for each sample with other samples and/or a standard level. Typically, the standard level is obtained by measuring the same marker or markers in the subject before drug administration. Depending upon the difference between the measured and standard levels, the drug can be considered to have an effect on cancer. If multiple biomarkers are measured, at least one and up to all of the biomarkers must change, in the expected direction, for the drug to be considered effective. Preferably, multiple markers must change for the drug to be considered effective, and preferably, such change is statistically significant.

As will be apparent to those of ordinary skill in the art, the above description is not limited to a candidate drug, but is applicable to determining whether any therapeutic intervention is effective in treating cancer.

In a typical embodiment, a subject population having cancer is selected for the study. The population is typically selected using standard protocols for selecting clinical trial subjects. For example, the subjects are generally healthy, are not taking other medication, and are evenly distributed in age and sex. The subject population can also be divided into multiple groups; for example, different sub-populations may be suffering from different types or different degrees of the disorder to which the candidate drug is addressed. The stratification of the patient population may be made based on the levels of biomarkers of the present invention.

In general, a number of statistical considerations must be made in designing the trial to ensure that statistically significant changes in biomarker measurements can be detected following drug administration. The amount of change in a biomarker depends upon a number of factors, including strength of the drug, dose of the drug, and treatment schedule. It will be apparent to one skilled in statistics how to determine appropriate subject population sizes. Preferably, the study is designed to detect relatively small effect sizes.

The subjects optionally may be “washed out” from any previous drug use for a suitable period of time. Washout removes effects of any previous medications so that an accurate baseline measurement can be taken. At time tₒ, a biological sample is obtained from each subject in the population. Next, an assay or variety of assays is performed on each subject's sample to measure levels of particular biomarkers of the invention. The assays can use conventional methods and reagents, as described above. If the sample is blood, then the assays typically are performed on either serum or plasma. For other fluids or tissues, additional sample preparation steps are included as necessary before the assays are performed. The assays measure values of at least one of the biological markers described herein. In some embodiments, only a single marker is monitored, while in other embodiments, a combination of factors, up to the total number of markers, is monitored. The markers may also be monitored in conjunction with other measurements and factors associated with cancer (e.g., MRI imaging). The number of biological markers whose values are measured depends upon, for example, the availability of assay reagents, biological fluid, and other resources.

Next, a predetermined dose of a candidate drug is administered to a portion or sub-population of the same subject population. Drug administration can follow any suitable schedule over any time period, and the sub-population can include some or all of the subjects in the population. In some cases, varying doses are administered to different subjects within the sub-population, or the drug is administered by different routes. Suitable doses and administration routes depend upon specific characteristics of the drug. At time t₁, after drug administration, another biological sample (the “t₁ sample”) is acquired from the sub-population. Typically, the sample is the same type of sample and processed in the same manner as the sample acquired from the subject population before drug administration (the “tₒ sample”). The same assays are performed on the t₁ sample as on the tₒ sample to obtain measurement values. Subsequent sample acquisitions and measurements can be performed as many times as desired over a range of times t₂ to tₙ.

Typically, a different sub-population of the subject population is used as a control group, to which a placebo is administered. The same procedure is then followed for the control group: obtaining the biological sample, processing the sample, and measuring the biological markers to obtain measurement values. Additionally, different drugs can be administered to any number of different sub-populations to compare the effects of the multiple drugs. As will be apparent to those of ordinary skill in the art, the above description is a highly simplified description of a method involving a clinical trial. Clinical trials have many more procedural requirements, and it is to be understood that the method is typically implemented following all such requirements.

Paired measurements of the various biomarkers are now available for each subject. The different measurement values are compared and analyzed to determine whether the biological markers changed in the expected direction for the drug group but not for the placebo group, indicating that the candidate drug is effective in treating the disease. In preferred embodiments, such change is statistically significant. The measurement values at time t₁ for the group that received the candidate drug are compared with standard measurement values, preferably the measured values before the drug was given to the group, i.e., at time tₒ. Typically, the comparison takes the form of statistical analysis of the measured values of the entire population before and after administration of the drug or placebo. Any conventional statistical method can be used to determine whether the changes in biological marker values are statistically significant. For example, paired comparisons can be made for each biomarker using either a parametric paired t-test or a non-parametric sign or sign rank test, depending upon the distribution of the data.

In addition, tests may be performed to ensure that statistically significant changes found in the drug group are not also found in the placebo group. Without such tests, it cannot be determined whether the observed changes occur in all patients and are therefore not a result of candidate drug administration.

As indicated in Table S5, some of the marker measurement values are higher in samples from cancer patients. A significant change in the appropriate direction in the measured value of one or more of the markers indicates that the drug is effective. If only one biomarker is measured, then that value must increase or decrease to indicate drug efficacy. If more than one biomarker is measured, then drug efficacy can be indicated by change in only one biomarker, all biomarkers, or any number in between. In some embodiments, multiple markers are measured, and drug efficacy is indicated by changes in multiple markers. Measurements can be of both biomarkers of the present invention and other measurements and factors associated with cancer (e.g., measurement of biomarkers reported in the literature and/or CT imaging). Furthermore, the amount of change in a biomarker level may be an indication of the relatively efficacy of the drug.

In addition to determining whether a particular drug is effective in treating cancer, biomarkers of the invention can also be used to examine dose effects of a candidate drug. There are a number of different ways that varying doses can be examined. For example, different doses of a drug can be administered to different subject populations, and measurements corresponding to each dose analyzed to determine if the differences in the inventive biomarkers before and after drug administration are significant. In this way, a minimal dose required to effect a change can be estimated. In addition, results from different doses can be compared with each other to determine how each biomarker behaves as a function of dose. Based on the results of drug screenings, the markers of the invention may be used as theragnostics; that is, they can be used to individualize medical treatment.

In another aspect, the invention provides a kit for detecting marker(s) of the present invention. The kit may be prepared as an assay system including any one of assay reagents, assay controls, protocols, exemplary assay results, or combinations of these components designed to provide the user with means to evaluate the expression level of the marker(s) of the present invention.

In another aspect, the invention provides a kit for diagnosing cancer in a patient including reagents for detecting at least one polypeptide or polynucleotide marker in a biological sample from a subject.

The kits of the invention may comprise one or more of the following: an antibody, wherein the antibody specifically binds with a marker, a labeled binding partner to the antibody, a solid phase upon which is immobilized the antibody or its binding partner, instructions on how to use the kit, and a label or insert indicating regulatory approval for diagnostic or therapeutic use.

The invention further includes microarrays comprising markers of the invention, or molecules, such as antibodies, which specifically bind to the markers of the present invention. In this aspect of the invention, standard techniques of microarray technology are utilized to assess expression of the polypeptides biomarkers and/or identify biological constituents that bind such polypeptides. Protein microarray technology is well known to those of ordinary skill in the art and is based on, but not limited to, obtaining an array of identified peptides or proteins on a fixed substrate, binding target molecules or biological constituents to the peptides, and evaluating such binding. Arrays that bind markers of the invention also can be used for diagnostic applications, such as for identifying subjects that have a condition characterized by expression of polypeptide biomarkers, e.g., cancer.

The assay system preferably also includes one or more controls. The controls may include: (i) a control sample for detecting sensitivity to a chemotherapeutic agent or agents being evaluated for use in a patient; (ii) a control sample for detecting resistance to the chemotherapeutic(s); (iii) information containing a predetermined control level of markers to be measured with regard to the chemotherapeutic sensitivity or resistance (e.g., a predetermined control level of a marker of the present invention that has been correlated with sensitivity to the chemotherapeutic(s) or resistance to the chemotherapeutic).

In another embodiment, a means for detecting the expression level of the marker(s) of the invention can generally be any type of reagent that can include, but are not limited to, antibodies and antigen binding fragments thereof, peptides, binding partners, aptamers, enzymes, and small molecules. Additional reagents useful for performing an assay using such means for detection can also be included, such as reagents for performing immunohistochemistry or another binding assay.

The means for detecting of the assay system of the present invention can be conjugated to a detectable tag or detectable label. Such a tag can be any suitable tag which allows for detection of the reagents used to detect the marker of interest and includes, but is not limited to, any composition or label detectable by spectroscopic, photochemical, electrical, optical or chemical means. Useful labels in the present invention include: biotin for staining with labeled streptavidin conjugate, magnetic beads (e.g., Dynabeads™), fluorescent dyes (e.g., fluorescein, texas red, rhodamine, green fluorescent protein, and the like), radiolabels ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads.

In addition, the means for detecting of the assay system of the present invention can be immobilized on a substrate. Such a substrate can include any suitable substrate for immobilization of a detection reagent such as would be used in any of the previously described methods of detection. Briefly, a substrate suitable for immobilization of a means for detecting includes any solid support, such as any solid organic, biopolymer or inorganic support that can form a bond with the means for detecting without significantly affecting the activity and/or ability of the detection means to detect the desired target molecule. Exemplary organic solid supports include polymers such as polystyrene, nylon, phenol-formaldehyde resins, and acrylic copolymers (e.g., polyacrylamide). The kit can also include suitable reagents for the detection of the reagent and/or for the labeling of positive or negative controls, wash solutions, dilution buffers and the like. The assay system can also include a set of written instructions for using the system and interpreting the results.

The assay system can also include a means for detecting a control marker that is characteristic of the cell type being sampled can generally be any type of reagent that can be used in a method of detecting the presence of a known marker (at the nucleic acid or protein level) in a sample, such as by a method for detecting the presence of a biomarker described previously herein. Specifically, the means is characterized in that it identifies a specific marker of the cell type being analyzed that positively identifies the cell type. For example, in a tumor assay, it is desirable to screen cancer cells for the level of the biomarker expression and/or biological activity. Therefore, the means for detecting a control marker identifies a marker that is characteristic of a cell, so that the cell is distinguished from other cell types, such as a connective tissue or inflammatory cells. Such a means increases the accuracy and specificity of the assay of the present invention. Such a means for detecting a control marker include, but are not limited to: a probe that hybridizes under stringent hybridization conditions to a nucleic acid molecule encoding a protein marker; PCR primers which amplify such a nucleic acid molecule; an aptamer that specifically binds to a conformationally-distinct site on the target molecule; and/or an antibody, antigen binding fragment thereof, or antigen binding peptide that selectively binds to the control marker in the sample. Nucleic acid and amino acid sequences for many cell markers are known in the art and can be used to produce such reagents for detection.

The assay systems and methods of the present invention can be used not only to identify patients that are predicted to survive or be responsive to treatment, but also to identify treatments that can improve the responsiveness of cancer cells which are resistant to treatment, and to develop adjuvant treatments that enhance the response of the treatment and survival.

The invention now being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention.

### EXAMPLES

### Example 1

This Example shows that RalA expression in human bladder urothelial carcinoma tissue is associated with poor patient survival.

A tissue microarray of bladder carcinomas (Smith et al 2009), stages pTa-T4, was stained with antibodies specific for RalA and RalB proteins and immunohistochemistry was performed as detailed below.

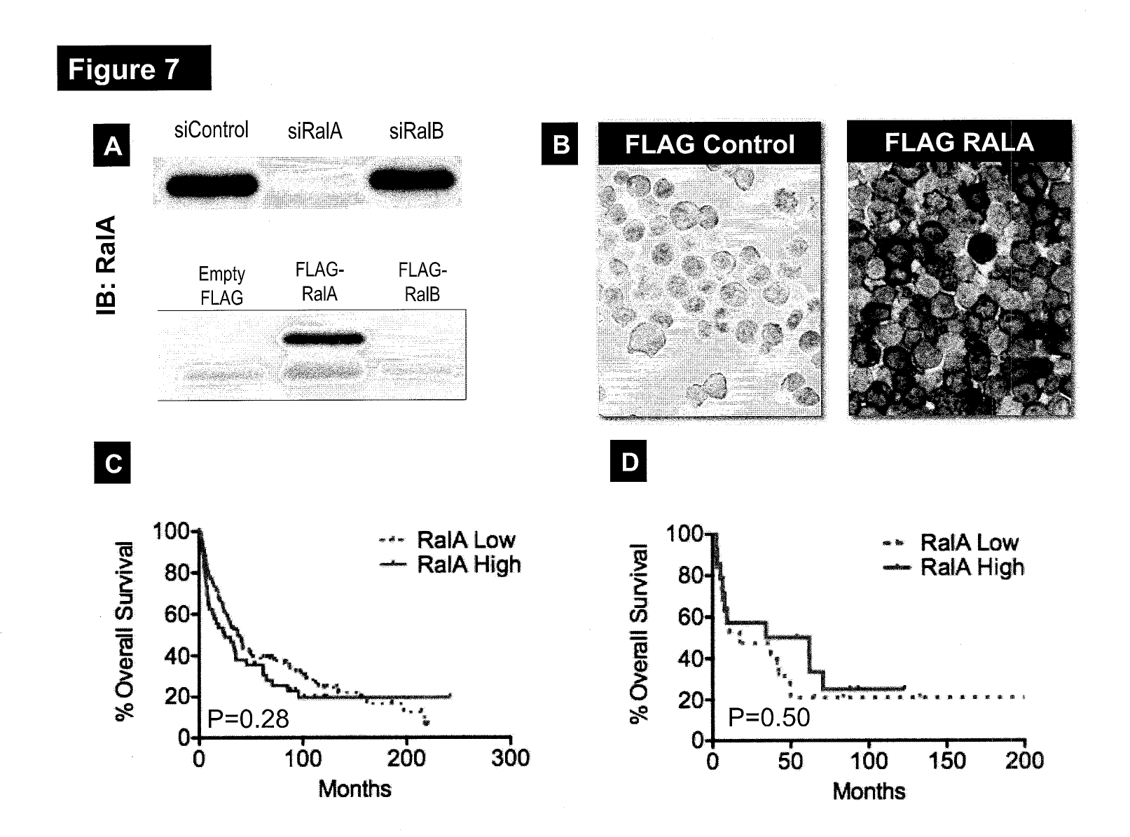

The specificity of the anti-RalA antibody (mouse monoclonal raised against a RalA-specific epitope (clone 8, BD Biosciences, Pharmingen, San Diego, Calif.)) is demonstrated in Figure S1A, demonstrating specific detection of depletion of RalA, but not RalB, by transient transfection of their respective siRNAs into UM-UC-3 bladder cancer cells, and confirmed by specific detection of transiently overexpressed FLAG-RalA, but not FLAG-RalB. (For reference, the identical lysates were blotted for RalB in Figure S2A. To test whether this antibody is capable of measuring expression of RalA in a semiquantitative manner by immunohistochemistry, we employed a cell line expressing relatively low endogenous RalA, UM-UC-3 (Smith et al 2007), stably overexpressing GFP (control) or GFP-tagged RalA (test). Cell lines were expanded in large format cell culture dishes under standard conditions (Titus et al 2005), pelleted, fixed by 10% formalin, and embedded in paraffin by standard methods. The staining protocol used DAKO Dual Endogenous Enzyme Block (DAKO North America, Carpinteria, Calif.) for 10 minutes, the RalA primary at 1:1600 dilution for 30 minutes in DAKO antibody diluent, and detection with DAKO Envision Dual Link secondary (30 minutes) and DAB+ chromogen (10 minutes) before hematoxylin counterstain. Slides were imaged in an Aperio XT whole slide digital scanner and photographed at 40× in ImageScope (both, Aperio Technologies, Inc., Vista, Calif.). It was found that the antibody detected low level endogenous expression of RalA, showing foci of membranous and cytoplasmic expression (FIG. 7B S1B), similar to prior reports regarding the subcellular localization of RalA (Frankel et al 2005). In contrast, cells overexpessing GFP-tagged RalA showed a high level of staining, supporting the premise that this detection method was capable of measuring expression of RalA in a semiquantitative fashion in the setting of immunohistochemistry.

For RalB, we used polyclonal antibody raised against RalB (R&D systems, Minneapolis, Minn.) and validated for specific detection of RalB. Figure S2A demonstrates the specificity of this antibody to RalB showing specific detection of depletion of RalB, but not RalA, by transient transfection of their respective siRNAs into UM-UC-3 bladder cancer cells, confirmed by specific detection of transiently overexpressed FLAG-RalB, but not FLAG-RalA. (For reference, the identical lysates were blotted for RalA and presented in Figure S1A) After microwave antigen retrieval, the RalB antibody was incubated at 1:400 dilution for 1 hour at room temperature with detection by immunoperoxidase reaction and DAB chromogen as before. Again for immunohistochemical workup we employed the UM-UC-3 cell line expressing relatively low endogenous RalB, stably overexpressing FLAG (control) or FLAG-tagged RalB (test). As with RalA, we observed semiquantitative detection of RalB expression using this protocol (Figure S2A-B).

Next, expression of RalA was evaluated in the human bladder carcinoma tissue microarray (Smith et al 2009) using the same staining protocol described above. We observed a similar pattern of focal membranous and cytoplasmic expression of RalA in the tissues evaluated, and scored expression of RalA semi quantitatively as either low (low to moderate intensity, or only focal higher expression in <50% of cells in cores examined) or high (strong, diffuse positive staining in >50% of cells in cores examined). We then used the Chi-Square test (implemented in Matlab Version 2010b, The Mathworks, Natick, Mass.), or Chi-Square test for trend, and Mantel-Cox Log Rank tests (Mantel-Haenszel Method, implemented in Prism, GraphPad Software, La Jolla, Calif.) to test the association between RalA low versus high staining with clinicopathologic parameters among tumors in the cohort.

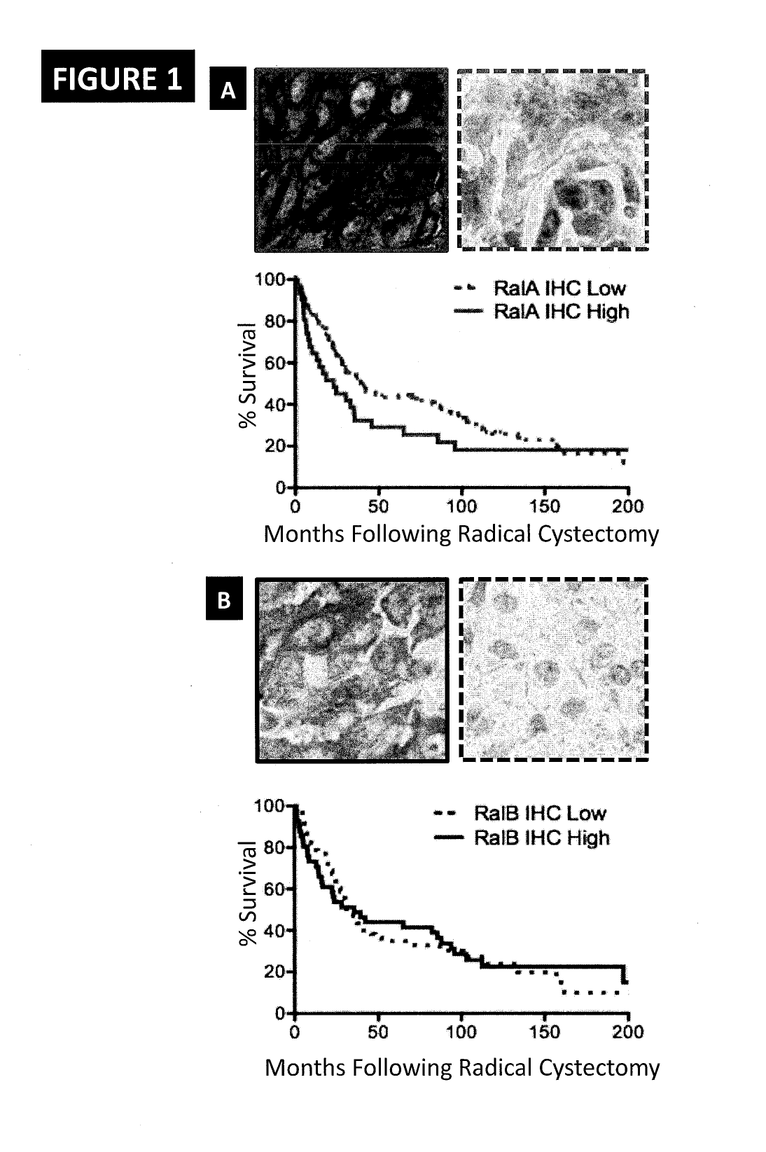

Table S1 summarizes results for RalA immunohistochemistry. Out of 145 cases (including urothelial carcinoma (N=110) and other less common histological variants (N=35)) where clinicopathologic and follow-up data were known, we observed evaluable RalA staining in 143. Of these, 98/143 (68.5%) were scored as RalA low, and 45/143 (31.5%) were scored as RalA high. We observed that RalA staining class was not significantly associated with pathologic stage, nodal status, gender, lymphovascular space invasion (LVSI), or presence of concomitant carcinoma in situ (CIS). RalA staining was significantly different among the main histologic types of bladder cancer, including squamous cell carcinomas, adenocarcinomas, and other rarer variants, P=0.028. As regards survival outcomes, a trend toward association between RalA expression and overall survival was observed most prominently in urothelial carcinoma cases (N=110, P=0.16, FIG. 1A), but was not significant when examining all bladder carcinomas (N=144, P=0.28) consistent with a nonsiginficant trend toward better survival in RalA high non-urothelial cases (N=34, P=0.50), see Figure S1C-D.

We then performed RalB immunohistochemical staining on the same microarray as for RalA. Table S2 summarizes results for RalB immunohistochemistry. Of 145 cases where clinicopathologic and follow-up data were known, we observed evaluable RalB staining in 137. Of these, 78/137 (56.9%) were scored as RalB low, and 59/137 (43.1%) were scored as RalB high. Again, we did not observe significant differences in RalB staining associated with pathologic stage, nodal status, gender, LVSI, CIS. RalB staining was significantly different among the main histologic types of bladder cancer P=0.018. However, RalB staining class was not significantly associated with overall survival in urothelial (N=104 P=0.99, FIG. 1B), all bladder carcinomas (N=137, P=0.48, Figure S2C) or non-urothelial cases (N=33, P=0.48 Figure S2D), both Mantel-Cox.

FIG. 1A shows representative photomicrographs of strong, diffuse RalA staining (RalA High, solid) and weak RalA staining (RalA Low, dashed). FIG. 1A further shows Kaplan-Meier analysis of overall survival, stratified by expression level of RalA, showing a non-significant trend in favor of poorer overall survival for cases expressing strong RalA (Log Rank P=0.16). Wilcoxon-Breslow testing of these curves, which weights early events, identified a significant difference (P=0.04). FIG. 1B shows similar micrographs. but for RalB showing strong diffuse staining (blue, solid) and weak RalB staining (blue, dashed), and Kaplan-Meier analysis for RalB expression finding non-significant difference by Log Rank or Wilcoxon-Breslow methods.

Finally, we tested the association between RalA and RalB in cases where staining was interpretable for both GTPases (N=136), finding only a nonsignificant trend between them (Table S3, P=0.11). To determine whether combinatorial evaluation of RalA and RalB staining might exceed the performance of either individually as regards stratification of overall survival among cases of urothelial bladder cancer (N=104), we stratified cases in three classes: RalA Low/RalB Low, RalA Low/RalB High & RalA High/RalB Low, or RalA High/RalB High. While the trend in survival was that of decreasing survival as a function of increased staining of either or both GTPases (Figure S3), it did not exceed the significance (log rank P=0.45 for trend) of that of RalA alone (P=0.16, Wilcoxon P=0.04, FIG. 1A).

### Example 2

This Example illustrates the identification of a common trascriptional signature of RalA and RAM in human bladder cancer cells.

Ral GTPases signal to gene expression through a variety of transcription factors (Neel et al 2011, Nitz et al 2011, Oxford et al 2007). Since tumors with the same levels of Ral protein but different levels of GTPase activation or effector interactions may induce such transcription factors to varying levels, which in turn might induce different clinical phenotypes, we hypothesized that Ral-dependent transcriptomic profiles might better capture pathway output and associate with salient clinicopathologic factors and outcomes. Accordingly, we developed a transcriptional signature of Ral pathway status based on profiling cells depleted of RalA or RalB. siRNA was used to deplete RalA or RalB from bladder cancer cells and the resultant transcriptional changes were profiled by microarray. Given the significant overlap between RalA and RalB-dependent transcriptional targets, a “core” signature of the transcriptional program common to both RalA and RalB was developed by choosing a union of 60 probesets regulated by RalA and RalB depletion in human bladder cancer cells (minimum 2 fold, >100 microarray expression units difference between closest replicates, Table S4), to which was applied the COXEN (co-expression extrapolation) principle (Lee et al 2007, Smith et al 2010) to define a subset of 39 probesets (Table S5) maintaining concordant expression in a published bladder cancer microarray cohort of patients treated by radical cystectomy (N=91) reported by Sanchez-Carbayo et al. (Sanchez-Carbayo et al 2006). This process of identification is described in detail below.

Despite key findings from in vitro and in vivo model systems (Chien and White 2003, Hamad et al 2002, Lim et al 2005, Lim et al 2006), little data from human tissues support the importance and relevance of Ral GTPases to human tumors. Given the fact that the Ral paralogs, RalA and RalB are known to regulate transcription through several pathways, reviewed in (Feig 2003), we hypothesized that transcription might serve as an integrated way to examine the status of this pathway in human tumors. To implement this strategy we used prior published dataset from our group, Oxford et al. (Oxford et al 2007), where we used siRNAs specifically depleting RalA or RalB GTPases in UM-UC-3 urothelial carcinoma cells for 72 hours, using siRNA to probe for transcriptional patterns dependent on these GTPases by profiling with Affymetrix HG-U133A high density oligonucleotide DNA microarrays. We found a significant overlap between RalA and RalB, and we found that such genes included both prior reported targets of RalA and RalB (Chien et al 2006, Hu and Mivechi 2003, Okan et al 2001) and important, novel mediators of cancer phenotypes, including CD24 (Smith et al 2006). Availing ourselves to these data, we evaluated these genes in a comprehensive fashion in microarray-profiled bladder cancer datasets.

To develop the above Ral data into a signature, we first processed and normalized the microarray data in two replicates of control siRNA (GL2, firefly luciferase, non-targeting); two replicates of siRalA; and two replicates of siRalB, using the technique of Robust Multichip Average (RMA) (Irizarry et at 2003), implemented in Matlab 8201013 (The Mathworks, Natick, Mass.), extracting Log 2 transformed expression values for the 22843 probes on the chip. All further analyses were implemented in Matlab, with additional use of Prism (GraphPad Software, La Jolla, Calif.) for plotting:

As a first criterion, we extracted a list of microarray probes regulated 2-fold (increased or decreased) by treatment with siRalA or siRalB. To screen against genes with artificially increased fold changes (due to lack of expression in either of the replicates), we used a second cutoff requiring that candidate Ral-dependent probes exhibit a minimum of 100 units (arbitrary expression values from the micoarray data) difference between replicates of siControl and siRal samples. These analyses resulted in 130 candidate probes regulated by RalA, 152 candidate probes regulated by RalB. Given our goal to generate a core signature of the transcriptional output of the Ral pathway to use to interrogate tumor samples, we used the intersect of RalA and RalB-regulated probes, a total of 60 probes (Table S4).

First, we wished to use hierarchical clustering to visualize relationships between samples across expression of Ral Signature genes in siControl, siRalA, and siRalB cell lines and a set of 91 urothelial carcinomas of varying pathologic stage profiled on the same Affymetrix HG-U133A platform by Sanchez-Carbayo et al. (Sanchez-Carbayo et al 2006), available as supplementary data on the publication's website. However, globally different patterns of gene expression between cell line and tumor models prevent facile clustering of such samples together (Lee et al 2007). To address this issue, we developed and reported an informatic technique, called Coexpression Extrapolation, or COXEN, to uncover subsets of these probes that maintain concordant expression between the damsels and excluding probes showing discordant or idiosyncratic expression as a function of being derived from profiling a cell line or tumor sample (Lee et al 2007, Williams et al 2009). For N probes, this technique uses a N by N-sized correlation matrix, recording correlation coefficient for each probe to all other candidate probes. Such a calculation is made for both the cell lines and the tumor samples in question. Then, each row of these correlation matrix is itself correlated, measuring a “correlation of correlations”—the COXEN Coefficient—that estimates the relative concordance of each probe to genes in either the cell line set or the tumor set. Probes showing a coefficient greater than an arbitrary threshold (generally 0) are considered concordant, while probes below such a threshold are excluded from further analysis, predictive model development, or final signature. After application of a COXEN coefficient cutoff to the 60 probes regulated >2-fold by RalA and RalB, we identified a final signature of Ral-dependent transcription comprising 39 probes, which are termed the Ral Transcriptional Signature, as listed in Table S5.

An important aspect of the COXEN methodology is that this analytic step, allowing exclusion of spurious cohort-specific (e.g., cell line versus tumor tissue) probes, is blinded to status or outcomes in the second dataset. For example, in our prior analyses dealing with prediction of chemotherapeutic response outcomes for human clinical trial patients based on cell line-derived signatures of drug sensitivity, all COXEN analyses and predictions were blinded to trial outcomes during candidate biomarker selection and or exclusion. Similarly, the clinicopathologic characteristics of the 91 urothelial cancers from the Sanchez-Carbayo et al. dataset (Sanchez-Carbayo et al 2006) are blinded throughout the concordant probe selection process.

### Example 3

This example shows that the Ral Signature characterizes invasive disease in human bladder cancer

Using the 39 aforementioned Ral signature probes, we clustered the 91 tumors described above (Sanchez-Carbayo et al 2006) with control or Ral-depleted cells and found that non-muscle invasive (stage pTa, pT1) tumors clustered with the Ral-depleted cells, while muscle invasive (stage T2+) tumors clustered with control treated cells (FIG. 2A). This result constitutes the first systematic and comprehensive demonstration of the importance of Ral GTPase-dependent transcription in any human tumor type.

To determine quantitatively if there is a relationship between tumor stage in this cohort and expression of the Ral signature, we used a weighted KNN classifier algorithm to classify the tumors based on similarity to Ral depleted cells (Ral Signature Negative, i.e., like siRalA and siRalB) or control cells (Ral Signature Positive, i.e., like control cells, expressing Ral and its transcriptional program). Our weighted KNN or “WNN” classifier has been reported in detail (Overdevest et al 2011, Smith et at 2011). Briefly, the weighted KNN classifier algorithm uses non-parametric (Spearman) correlation as distance metric to measure similarity of expression of Ral signature genes to Control or Ral-depleted cells, outputting a prediction score, which we call the “Ral Signature Score,” ranging from 0 to 1. This WNN classification algorithm, Matlab code available on request, was used to score the Ral signature in the Sanchez-Carbayo et al. samples as well as all other datasets examined. Using this approach, we observed a significant difference in distributions of Ral signature scores between non-muscle invasive bladder cancers (NMIBC) and muscle invasive bladder cancers (MIBC), P<0.0001 (FIG. 2B), where NMIBCs had lower Ral signature scores and MIBCs had higher Ral signature scores. Importantly, we used thousand-fold random permutation testing to examine the significance of our approach, confirming that this degree of difference was only associated with a 0.1% false discovery rate, confirmatory of the importance of Ral signature genes as opposed to global transcriptional differences between NMIBC and MIBC.

Finally, application of this signature to classify tumors of four additional independent cohorts of bladder tumors (Dyrskjot et at 2003, Kim et at 2010, Lindgren et at 2010, Stransky et al 2006) profiled on four different microarray platforms (total N=410) showed similar results (FIG. 2C-D, FIG. 54A-B).

### Example 4

This example describes the cross-microarray platform outcome predictions using the Ral Signature

Classification of tumors as Ral Transcriptional Signature Positive or Negative is straightforward in cases where both the cell lines used for prediction and tumors tested were profiled on the same platform (the Sanchez-Carbayo et al. cohort). However, given the multitude of microarray platforms available for use to study cancer, a means for cross-platform comparisons of gene expression is necessary for testing new cohorts. Additionally, if successful, such comparisons lend additional credibility to the phenomenon studied, showing its robustness of association with clinical characteristics across cohorts derived from different institutions, populations, ethnicities, etc. To test the Ral signature across platforms, generally we used Unigene cluster ID as a common identifier for transcripts (exceptions delineated below). In cases within the cell line training data where multiple Ral Signature probes represented the same Unigene cluster, zscored log 2 expression values were averaged. In the test dataset, if multiple probes represented the same Unigene cluster of interest, the probe with the highest median intensity was selected. Then the expression data, condensed to a single set of expression values for each Unigene, were used to predict Ral signature status. Between platforms, zscore-normalized data were used for cohorts where proportions of the clinicopathologic character of interest was represented in roughly equal proportions, while for cases where the characteristics of interest was represented in only a substantially skewed proportion of cases (e.g., the siControl (2 samples, 33.3%) and siRal cell line data (2 RalA and 2 RalB, 66.7%), group weighted zscores were used for normalization, as reported before (Smith et al 2011).

### Cross-Platform Implementation for Additional Bladder Cancer Microarray Cohorts

For the Dyrskjot et al. cohort (Dyrskjot et al 2003), data were downloaded from NCBI GEO (GSE88, GSE89) and Unigene annotations provided by Affymetrix used for mapping from U133A to HUGENE FL platforms. For the Stransky et al. cohort (Stransky et at 2006), Affymetrix annotation data for Unigene clusters were used to map the U133A data from the cell lines above to the U95AV2 data, downloaded from ArrayExpress (F-TABM-147). For the Kim et al. cohort (Kim et al 2010), high-quality Unigene cluster ID annotations were provided by ReMOAT (Barbosa-Morais et al. (Barbosa-Morais et al)) for the Illumina Chip platform data, which was downloaded from NCBI GEO (GSE13507). For the Dyrskjot et al. non-muscle invasive urothelial cancer progression dataset (Dyrskjot et al 2005), data and annotations were used as supplied by the publication's online supplement (www.mdl.dk) at the Aarhus University website. For the Lindgren et al. cohort (Lindgren et at 2010), data and annotations were downloaded from NCBI GEO (GSE19915), though in this case, HUGO gene symbols were used to map between the U133A and custom/normalized platforms.

### Cross-Platform Implementation for Squamous Cell Carcinoma Microarray Cohorts

We employed the same signature genes as in the bladder analysis, using a COXEN step with identical >0 cutoff as used for the bladder analyses. The first dataset, used for the COXEN step, was a published cohort of matched normal and malignant cases (N=53) by Su et al. (Su et al), profiled on HG-U133A (downloaded from NCBI GEO, GSE23400), resulting in a concordant set of 40 probes. Using these probes, predictions were made by the same methodology as the bladder cohorts, and Ral signature scores were compared between matched normal mucosae and squamous cancers (Wilcoxon Matched Pairs test, in Prism). For additional validation, a second set, profiled by Ye et al. on the Affymetrix HG-U133 Plus 2.0 platform (Ye et al 2008), downloaded from NCBI (GSE9844).

### Cross-Platform Implementation for Prostate Cancer Cohorts

Given prior findings associating androgen withdrawal with induction of Ral and transcription of VEGFC (Rinaldo et al 2006), we examined the Ral signature in a recently published dataset of microarray profiled, microdissected androgen dependent (N=10) and androgen independent (N=10) primary prostate tumors by Best et al. (Best et al 2005), downloaded from NCBI GEO (GSE2443). Using the same RalA and RalB regulated probes, we applied a COXEN step, as before, between the cell line data and the Best et al. cohort, profiled on the Affymetrix FIG-U133A platform, to uncover a concordant subset (COXEN coefficient cutoff >0) of 47 probes. These probes were used for analysis of subsequent cohorts below, using the WNN classifier to assign a Ral signature score as above.

For examination of the Ral signature in the in vitro LNCAP cohort by D'Antonio et al. (D'Antonio et al 2008) and the xenograft cohort by Terada et al. (Terada et al 2010), both profiled on the Affymetrix HG-U133 Plus 2.0 array, the 47 concordant probes were shared between both platforms and predications made as before. For the additional human cohort by Wei et al. (Wei et al 2007), which used a custom cDNA cohybridization microarray platform (Wei et al 2007), we first used KNN imputation (knnimpute command in Matlab, set to 3 nearest neighbors) to impute data missing due to nonexpression in the reference RNA case. Then, as above, we used Unigene ID to map the genes from this platform to the Affymetrix U133A platform used for the cell lines of the Ral signature for comparison.

Finally, as several reports have suggested a role for RalA or RalB in aggressive and metastatic phenotypes for prostate cancer (Oxford et al 2005, Wu et al 2010, Yin et al 2007), we wished to test for associations between the Ral signature and key aggression parameters, including seminal vesicle invasion, biochemical recurrence, and disease specific mortality. For these analyses we used two published gene expression profiled cohorts by Taylor et al. (Taylor et al 2010) (N=131 primary tumors at prostatectomy, profiled on Affymetrix Human Exon 1.0 ST Array, GSE21034) and Sboner et al. (Sboner et at 2010) (N=281 transurethral resections with incidental/limited disease profiled on the Illumina Human 6 k Transcriptionally Informative Gene Panel DASL Platform, GSE16560). For the Taylor et al. cohort, we used IDconverter (Alibes et al 2007), to extract gene symbols for the whole transcript summary data. These were then mapped to symbols for Ral Signature Genes the Affymetrix U133A platform to use for intermicroarray predictions and comparisons. For the Sboner et al. cohort, gene symbols provided in the GEO array annotation file were employed for inter-microarray comparisons and WNN predictions as above.

### Statistical Analysis of Ral Signature Score Distributions

In each case, predictions were made by the WNN algorithm outputting Ral signature scores for each case, which by definition vary between 0 (most like siRalA and siRalB depleted cells, i.e., signature negative) and 1 (most like siControl Ral-intact, i.e., signature positive). Scores were plotted in Prism (GraphPad Software), and differences in distributions of scores between relevant groups (e.g., pTa/T1 versus pT2+) tested by the Mann-Whitney U-test, Wilcoxon Matched Pairs Test (paired SCC cohort), the receiver operating characteristic, or Kruskal-Wallis test, as appropriate and indicated in the results section. For bladder cohorts where follow-up data were available (Sanchez-Carbayo et al. and Dyrsjøt et al. (Dyrskjot et at 2005, Sanchez-Carbayo et at 2006), signatures scores <0.5 were considered “Signature Negative” and scores >0.5 considered “Signature Positive.” Kaplan-Meier curves plotted by signature positive or negative were plotted in Prism, and differences in survival curves tested by the Log Rank or Wilcoxon-Breslow tests, as indicated. For the prostate cancer cohorts by Taylor et al. and Sboner et al., the 0.5 cutoff did not significantly stratify cases by biochemical recurrence free or disease free survival, instead both Kaplan-Meier curves were plotted at their optimal discriminating point, as indicated in the results section.

An important additional test of the significance of our approach is random permutation testing, testing the likelihood that such results could be observed by chance alone, by “false discovery” (Tsai et at 2003). These tests were performed for each initial cohort of bladder (Sanchez-Carbayo et al.), squamous (Su et al.), and prostate cancers (Best et al.), each of which were used for the COXEN step and implementation of the Ral signature and its assessment. For each of these cases, the significance (nominal Pvalue for difference in Ral signature scores between classes) was compared against 1000 random selections of microarray probes, which were each used as a “mock signature” sampling the background ability of random genesets to discriminate between classes of tumors (e.g., between non-muscle invasive and invasive bladder cancers). Of 1000 random genesets, only 1 equaled or exceeded the Ral signature for discriminating between non-muscle invasive and muscle invasive tumors (false discovery rate 0.1%), only 5 equaled or exceeded the Ral signature for discriminating between normal mucosa and squamous cell carcinoma (false discovery rate 0.5%), and only 11 equaled or exceeded the signature for discriminating between androgen dependent and independent prostate cancers (false discovery rate 1.1%).

### Example 5

This example shows that bladder cancer cells with metastatic and stem cell characteristics have high Ral Signature Scores

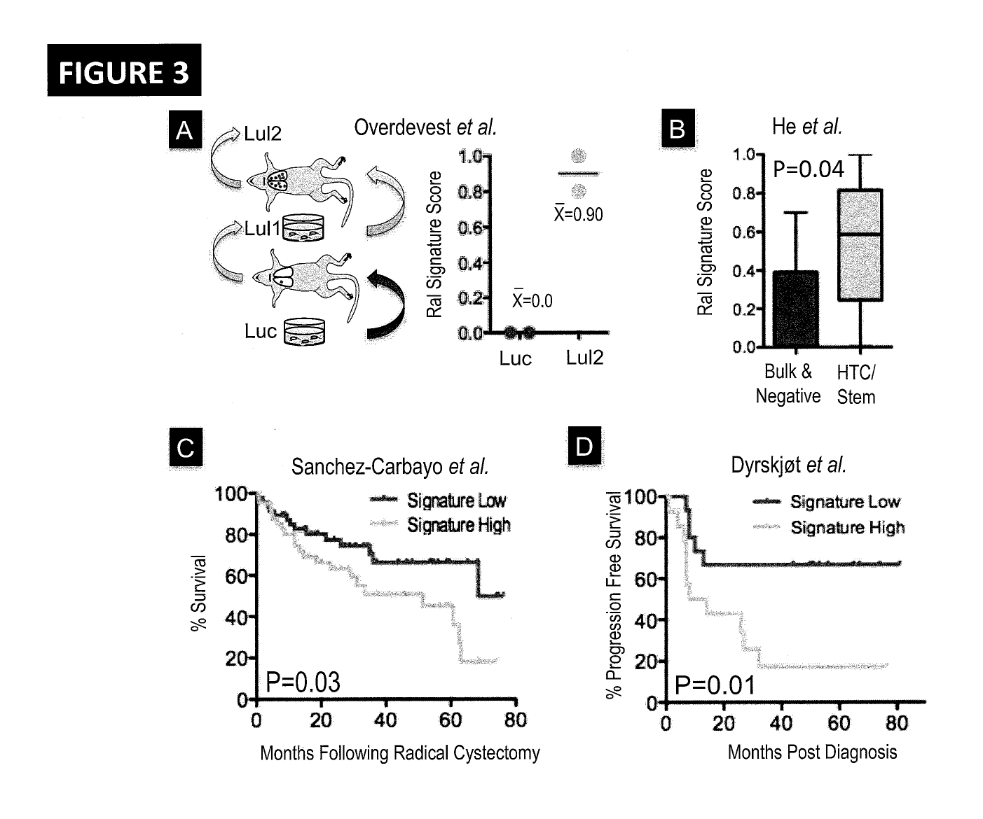

Given the correlation of Ral Signature scores with stage in bladder cancer patients, we next determined if the Ral Signature correlated with development of metastasis after surgery. We have recently developed a mouse model of lung metastasis using parental, poorly metastatic UM-UC-3 human bladder cancer cells. UM-UC-3 cells stably expressing firefly luciferase for bioluminescent imaging (Luc) were serially inoculated via tail vein to generate progressively more metastatic variants (Lul1 and Lul2) (FIG. 3A) which were then transcriptionally profiled. Lul2 was found to have a higher Ral Signature score than Luc cells, suggesting a role for Ral in bladder tumor progression, (FIG. 3A). Another report used fluorescence activated cell sorting to isolate an aggressive, highly tumorigenic/stem cell-like population of cells from SW780 bladder cancer cells, which were subsequently profiled by microarray (He et al 2009). The Ral signature score was higher in highly tumorigenic/stem cell-like SW780 isolates compared to parental and negative sorted populations, suggesting a role for Ral in the stem cell phenotype (FIG. 313).

### Example 6

This Example shows Ral Signature score can serve as a prognostic tool in human bladder cancer, as it is associated with poor patient survival and disease progression.

We investigated the relationship between Ral Signature score in tumors and patient survival in the Sanchez-Carbayo et al. cohort (FIG. 3C). Using a Ral Signature score cutoff of >0.5 or <0.5 to classify as signature positive or negative, respectively, we found that the Signature score significantly stratified cases by survival, with signature positive cases showing significantly worse survival (P=0.03, Log Rank), though this difference was not independent of the association of scores with stage in multivariate Cox models (P=0.57).

Furthermore, several groups have reported that non-muscle invasive (Ta and T1 stage) tumors that subsequently progress to Muscle invasion exhibit, a priori, the molecular characteristics of muscle invasive tumors (Dyrskjot et al 2003, Lindgren et al 2010, Wang et al 2009). Based on these observations and our findings of the Ral signature regarding invasion described above, we hypothesized that the Ral signature might be prognostic of subsequent progression in such cases. Using two published microarray cohorts of NMIBCs where progression during follow-up was documented (Dyrskjot et al 2005, Lindgren et al 2010) we evaluated the Ral signature score with respect to progression to muscle invasive stage disease. We found that the score significantly stratified progression free survival in a series (N=29) by Dyrskjøt et al. (FIG. 3D, P=0.01), while scores differed significantly between cases with and without progression in a second series reported by Lindgren et al. (N=97), (P=0.04, Figure S4).

### Example 7

This Example shows that the human squamous cell carcinoma has a lower Ral Signature score than normal mucosa

Recent reports suggest that Ral may play a tumor suppressor role in squamous cell carcinoma (SCC) (Sowalsky et al 2010, Sowalsky et al 2011). Hence, we reasoned that if these data have clinical significance, the Ral signature score should be lower in invasive SCCs as compared to normal squamous mucosa. We evaluated the signature in a published cohort of matched SCCs and histologically normal adjacent mucosae of the esophagus evaluated by microarray (Su et al.) as described below.

The same signature genes as in the bladder analysis were employed, using a COXEN step with identical >0 cutoff as used for the bladder analyses described in previous examples. The first dataset, used for the COXEN step, was a published cohort of matched normal and malignant cases (N=53) by Su et al. (Su et al), profiled on HG-U133A (downloaded from NCBI GEO, GSE23400), resulting in a concordant set of 40 probes. Using these probes, predictions were made by the same methodology as the bladder cohorts, and Ral signature scores were compared between matched normal mucosae and squamous cancers (Wilcoxon Matched Pairs test, in Prism).

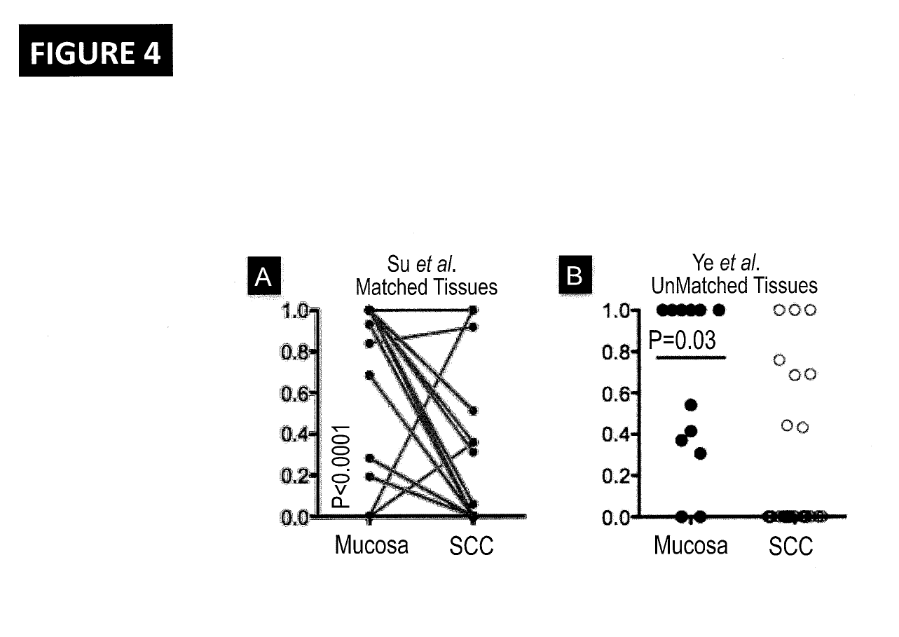

Strikingly, we found a significantly lower Ral signature score in normal mucosae compared to SCCs (FIG. 4A, P<0.0001). The significance of this difference over background differences in gene expression was tested by random permutation testing, observing a false discovery rate of 0.5%, supportive of the importance of Ral transcriptional targets.

The signature was then tested in a second, smaller cohort of oral SCCs (N=26) profiled by Ye et al 2008 on the Affymetrix HG-U133 Plus 2.0 platform (Ye et al 2008) downloaded from NCBI (GSE9844), as compared to normal mucosae (N=12). Significant difference in signature score distributions was found between normal and cancer cells (FIG. 413, P=0.03).

### Example 8

This Example shows that Ral Signature is present in the progression of prostatic adenocarcinoma

In animal models of prostate cancer RalA and/or RalB have been associated with metastasis and androgen independence (Rinaldo et al 2006, Ward et at 2001, Wu et al 2010, Yin et al 2007). We thus examined the status of the Ral signature with respect to important clinicopathologic surrogates of tumor aggressiveness in two recently-published, large patient cohorts (Sboner et al 2010, Taylor et al 2010).

In patients treated by radical prostatectomy (N=131, (Taylor et al 2010)), we did not observe significant correlations between the Ral signature scores and Gleason grade at biopsy (r=0.11, P=0.19) or prostatectomy (r=0.05, P=0.53), or with pathologic stage (P=0.86). However, Ral signature scores could risk stratify patients as a function of biochemical recurrence (P=0.05, FIG. 5A). Analogous to the results described regarding invasion in bladder cancer, Ral signature scores were significantly higher in cases showing seminal vesicle invasion, a poor prognostic factor (P=0.028, FIG. 5B).

We extended and generalized these findings by evaluating the Ral signature score on data from the Swedish Watchful Waiting Cohort (N=281) (Sboner et at 2010). In this cohort, cases were incidentally diagnosed on transurethral resection (clinical T1a-b), and managed with observation only over a 10 year period. Ral signature score was significantly correlated with Gleason score (r=0.13, P=0.03) and could stratify these cases by disease specific survival (P=0.03, FIG. 5C). Ral signature scores were not significantly associated with the TMPRSS-ERG fusion (Tomlins et al 2005) in this cohort (P=0.77).

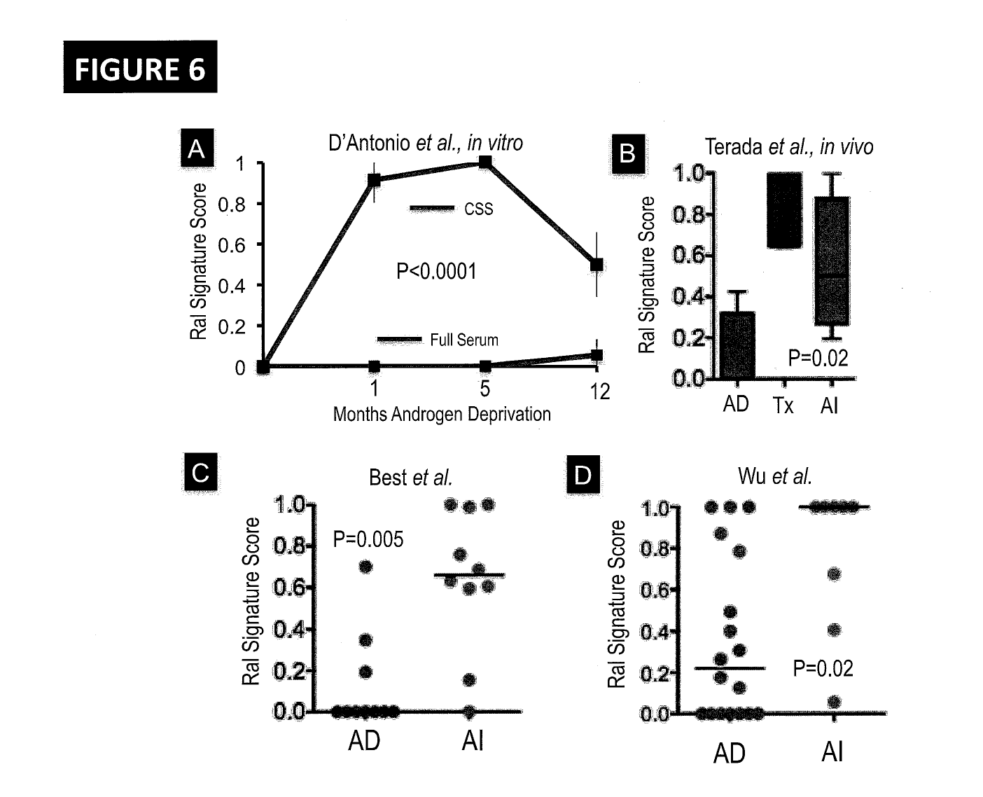

A clinically important dimension of prostate cancer biology is the issue of androgen dependence of disease (Tomlins et al 2007), in which a recent report has functionally implicated RalA through induction of VEGFC upon androgen withdrawal (Rinaldo et al 2006). To examine whether this was associated with changes in the Ral signature score through long-term androgen withdrawal, as occurs during therapy, we used a published gene expression study of longitudinal (1 year) in vitro androgen deprivation of LNCAP cells (D'Antonio et al 2008). Comparing the Ral signature scores of replicate androgen deprived cells to control cells over time, we observed an induction of the Ral signature scores over time (FIG. 6A, P<0.0001). Next, we examined an explanted tumor xenograft model of androgen independent progression of prostate cancer, KUCaP-2, which has been transcriptionally profiled at baseline, at their growth nadir upon castration, and upon androgen independent regrowth (Terada et al 2010). We found an induction of the Ral signature score over time that paralleled that observed in the LNCAP in vitro model (FIG. 6B).

To determine whether such a mechanism operated in human tumors, we examined the Ral signature score in a dataset of microarray profiled, microdissected androgen dependent (N=10) and androgen independent (N=10) primary prostate tumors (Best et al 2005, downloaded from NCBI GEO (GSE2443). We observed that the Ral signature score distributions differed significantly, with higher scores in androgen independent disease (FIG. 5A, P=0.005). Random permutation testing suggested that the observed degree of difference between androgen dependent and independent cases was specific to Ral rather than global differences in transcription (false discovery rate 1%). These findings were also generalized to a second cohort (Wei et al 2007) of androgen dependent (N=18) and androgen independent cases (N=18), profiled on a different, custom microarray platform (FIG. 5B, P=0.02).

The foregoing description of the present invention has been presented for purposes of illustration and description. Furthermore, the description is not intended to limit the invention to the form disclosed herein. Consequently, variations and modifications commensurate with the above teachings, and the skill or knowledge of the relevant art, are within the scope of the present invention. The embodiments described hereinabove are further intended to explain the best mode known for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art.

### REFERENCES

Alibes A, Yankilevich P, Canada A, Diaz-Uriarte R (2007). IDconverter and IDClight: conversion and annotation of gene and protein IDs. BMC Bioinformatics 8:9.
Amin M B (2009). Histological variants of urothelial carcinoma: diagnostic, therapeutic and prognostic implications. Mod Pathol 22 Suppl 2: S96-S118.
Atiya A F (2005). Estimating the Posterior Probabilities Using the K-Nearest Neighbor Rule. Neural Computation 17: 731-740.
Barbosa-Morais N L, Dunning M J, Samarajiwa S A, Darot I F, Ritchie M E, Lynch A G et al A re-annotation pipeline for Illumina BeadArrays: improving the interpretation of gene expression data. Nucleic Acids Res 38: e17.
Best C J, Gillespie J W, Yi Y, Chandramouli G V, Perlmutter M A, Gathright Y et al (2005). Molecular alterations in primary prostate cancer after androgen ablation therapy. Clin Cancer Res 11: 6823-6834.
Bodemann B O, White M A (2008). Ral GTPases and cancer: linchpin support of the tumorigenic platform. Nat Rev Cancer 8: 133-140.
Chan K S, Espinosa I, Chao M, Wong D, Ailles L, Diehn M et al (2009). Identification, molecular characterization, clinical prognosis, and therapeutic targeting of human bladder tumor-initiating cells. Proc Natl Acad Sci USA 106: 14016-14021.
Chien Y, White M A (2003). RAL GTPases are linchpin modulators of human tumour-cell proliferation and survival. EMBO Rep 4: 800-806.
Chien Y, Kim S, Bumeister R, Loo Y M, Kwon S W, Johnson C L et al (2006). RalB GTPase-mediated activation of the IkappaB family kinase TBK1 couples innate immune signaling to tumor cell survival. Cell 127: 157-170.

D'Antonio J M, Ma C, Monzon F A, Pflug B R (2008). Longitudinal analysis of androgen deprivation of prostate cancer cells identifies pathways to androgen independence. Prostate 68: 698-714.
Dyrskjot L, Thykjaer T, Kruhoffer M, Jensen J L, Marcussen N, Hamilton-Dutoit S et al (2003). Identifying distinct classes of bladder carcinoma using microarrays. Nat Genet 33: 90-96.
Dyrskjot L, Zieger K, Kruhoffer M, Thykjaer T, Jensen J L, Primdahl H et al (2005). A molecular signature in superficial bladder carcinoma predicts clinical outcome. Clin Cancer Res 11: 4029-4036.
Frankel P, Aronheim A, Kavanagh E, Balda M S, Matter K, Bunney T D et at (2005). RalA interacts with ZONAB in a cell density-dependent manner and regulates its transcriptional activity. Embo J 24: 54-62.
Feig L A (2003). Ral-GTPases: approaching their 15 minutes of fame. Trends Cell Riot 13: 419-425.
Feldmann G, Mishra A, Hong S M, Bisht S, Strock C J, Ball D W et al (2010). Inhibiting the cyclindependent kinase CDK5 blocks pancreatic cancer formation and progression through the suppression of Ras-Ral signaling. Cancer Res 70: 4460-4469.
Gildea J J, Harding M A, Seraj M J, Guiding K M, Theodorescu D (2002). The role of Ral A in epidermal growth factor receptor-regulated cell motility. Cancer Res 62: 982-985.
Hamad N M, Elconin J H, Karnoub A E, Bai W, Rich J N, Abraham R T et at (2002). Distinct requirements for Ras oncogenesis in human versus mouse cells. Genes Dev 16: 2045-2057.
He X, Marchionni L, Hansel D E, Yu W, Sood A, Yang J et al (2009). Differentiation of a highly tumorigenic basal cell compartment in urothelial carcinoma. Stem Cells 27: 1487-1495.
Henry D O, Moskalenko S A, Kaur K J, Fu M, Pestell R G, Camonis J H et al (2000). Ral GTPases contribute to regulation of cyclin D1 through activation of NF-kappaB. Mol Cell Blot 20: 8084-8092.
Hu Y, Mivechi N F (2003). HSF-1 interacts with Ral-binding protein 1 in a stress responsive, multiprotein complex with HSP90 in vivo. J Biol Chem 278: 17299-17306.
Irizarry R A, Bolstad B M, Collin F, Cope L M, Hobbs B, Speed T P (2003). Summaries of Affymetrix GeneChip probe level data. Nucleic Acids Res 31: e15.
Kan Z, Jaiswal B S, Stinson J, Janakiraman V, Bhatt D, Stern H M et al (2010). Diverse somatic mutation patterns and pathway alterations in human cancers. Nature 466: 869-873.
Kim W J, Kim E J, Kim S K, Kim Y J, Ha Y S, Jeong P et al (2010). Predictive value of progression related gene classifier in primary non-muscle invasive bladder cancer. Mol Cancer 9:3.
Lee J K, Havaleshko D M, Cho H, Weinstein I N, Kaldjian E P, Karpovich J et al (2007). A strategy for predicting the chemosensitivity of human cancers and its application to drug discovery. Proc Natl Acad Sci USA 104: 13086-13091.
Lim K H, Baines A T, Fiordalisi J J, Shipitsin M, Feig L A, Cox A D et al (2005). Activation of RalA is critical for Ras-induced tumorigenesis of human cells. Cancer Cell 7: 533-5.45.
Lim K H, O'Hayer K, Adam S J, Kendall S D, Campbell P M, Der C J et al (2006). Divergent roles for RalA and RalB in malignant growth of human pancreatic carcinoma cells. Curr Biol 16: 2385-2394.
Lindgren D, Frigyesi A, Gudjonsson S, Sjodahl G, Hallden C, Chebil G et al (2010). Combined gene expression and genomic profiling define two intrinsic molecular subtypes of urothelial carcinoma and gene signatures for molecular grading and outcome. Cancer Res 70: 3463-3472.
Neel N F, Martin T D, Stratford J K, Zand T P, Reiner D J, Der C J (2011). The RalGEF-Ral Effector Signaling Network: The Road Less Traveled for Anti-Ras Drug Discovery. Genes Cancer 2:275-287.
Nitz M D, Harding M A, Smith S C, Thomas S, Theodorescu D (2011). RREB1 Transcription Factor Splice Variants in Urologic Cancer. Am J Pathol 179: 477-486.
Okan E, Drewett V, Shaw P E, Jones P (2001). The small-GTPase RalA activates transcription of the urokinase plasminogen activator receptor (uPAR) gene via an API-dependent mechanism. Oncogene 20: 1816-1824.
Overdevest J B, Thomas S, Kristiansen G, Hansel D E, Smith S C, Theodorescu D (2011). CD24 offers a therapeutic target for control of bladder cancer metastasis based on a requirement for lung colonization. Cancer Res.
Oxford G, Owens C R, Titus B J, Foreman T L, Herlevsen M C, Smith S C et al (2005). RalA and RalB: antagonistic relatives in cancer cell migration. Cancer Res 65: 7111-7120.
Oxford G, Smith S C, Hampton G, Theodorescu D (2007). Expression profiling of Ral-depleted bladder cancer cells identifies RREB-1 as a novel transcriptional Ral effector. Oncogene 26:7143-7152.
Rangarajan A, Hong S J, Gifford A, Weinberg R A (2004). Species- and cell type-specific requirements for cellular transformation. Cancer Cell 6: 171-183.
Rinaldo F, Li J, Wang E, Muders M, Datta K (2006). RalA regulates vascular endothelial growth factor-C(VEGF-C) synthesis in prostate cancer cells during androgen ablation. Oncogene.
Rosse C, Hatzoglou A, Parrini M C, White M A, Chavrier P, Camonis 1 (2006). RalB mobilizes the exocyst to drive cell migration. Mol Cell Biol 26: 727-734.
Sablina A A, Chen W, Arroyo J D, Corral L, Elector M, Bulmer S E et al (2007). The tumor suppressor PP2A Abeta regulates the RalA GTPase. Cell 129: 969-982.
Sanchez-Carbayo M, Socci N D, Lozano 0.1, Saint F, Cordon-Cardo C (2006). Defining molecular profiles of poor outcome in patients with invasive bladder cancer using oligonucleotide microarrays. J Clin Oncol 24: 778-789.
Sboner A, Demichelis F, Calza S, Pawitan Y, Setlur S R, Hoshida Y et al (2010). Molecular sampling of prostate cancer: a dilemma for predicting disease progression. BMC Med Genomics 3:8.
Smith S C, Oxford G, Wu Z, Nitz M D, Conaway M, Frierson H F et al (2006). The metastasis associated gene CD24 is regulated by Ral GTPase and is a mediator of cell proliferation and survival in human cancer. Cancer Res 66: 1917-1922.
Smith S C, Oxford G, Boras A S, Owens C, Havaleshko D, Brautigan D L et al (2007). Expression of ral GTPases, their effectors, and activators in human bladder cancer. Clin Cancer Res 13: 3803-3813.
Smith S C, Nicholson B, Nitz M, Frierson H F, Jr., Smolkin M, Hampton G et al (2009). Profiling Bladder Cancer Organ Site-Specific Metastasis Identifies LAMC2 as a Novel Biomarker of Hematogenous Dissemination. Am J Pathol 174: 371-379.
Smith S C, Theodorescu D (2009). The Ral GTPase pathway in metastatic bladder cancer: key mediator and therapeutic target. Urol Oncol 27: 42-47.
Smith S C, Baras A S, Lee J K, Theodorescu D (2010). The COXEN principle: translating signatures of in vitro chemosensitivity into tools for clinical outcome prediction and drug discovery in cancer. Cancer Res 70: 1753-1758.
Smith S C, Baras A S, Dancik G, Ru Y, Ding K F, Moskaluk C A et al (2011). A 20-gene model for molecular nodal staging of bladder cancer: development and prospective assessment. Lancet Oncol 12: 137-143.
Sowalsky A G, Alt-Holland A, Shamis Y, Garlick J A, Feig L A (2010). RalA suppresses early stages of Ras-induced squamous cell carcinoma progression. Oncogene 29: 45-55.
Sowalsky A G, Alt-Holland A, Shamis Y, Garlick J A, Feig L A (2011). RalA function in dermal fibroblasts is required for the progression of squamous cell carcinoma of the skin. Cancer Res 71: 758-767.
Stransky N, Vallot C, Reyal F, Bernard-Pierrot I, de Medina S G, Segraves R et al (2006). Regional copy number-independent deregulation of transcription in cancer. Nat Genet 38: 1386-1396.
Su H, Hu N, Yang H H, Wang C, Takikita M, Wang Q H et al Global Gene Expression Profiling and Validation in Esophageal Squamous Cell Carcinoma and its Association with Clinical Phenotypes. Clin Cancer Res.
Suzuki J, Yamazaki Y, Li G, Kaziro Y, Koide (2000). Involvement of Ras and Ral in chemotactic migration of skeletal myoblasts. Mol Cell Biol 20: 4658-4665.
Taylor B S, Schultz N, Hieronymus H, Gopalan A, Xiao Y, Carver B S et al (2010). Integrative genomic profiling of human prostate cancer. Cancer Cell 18: 11-22.
Tchevkina E, Agapova L, Dyakova N, Martinjuk A, Komelkov A, Tatosyan A (2005). The small G-protein RalA stimulates metastasis of transformed cells. Oncogene 24: 329-335.
Terada N, Shimizu Y, Kamba T, Inoue T, Maeno A, Kobayashi T et al (2010). Identification of EP4 as a potential target for the treatment of castration-resistant prostate cancer using a novel xenograft model. Cancer Res 70: 1606-1615.
Titus B, Frierson H F, Jr., Conaway M, Ching K, Guise T, Chirgwin J et al (2005). Endothelin axis is a target of the lung metastasis suppressor gene RhoGD12. Cancer Res 65: 7320-7327.
Tsai C A, Chen Y J, Chen J J (2003). Testing for differentially expressed genes with microarray data. Nucleic Acids Res 31: e52.
Tomlins S A, Rhodes D R, Perner S, Dhanasekaran S M, Mehra R, Sun X W et al (2005). Recurrent fusion of TMPRSS2 and ETS transcription factor genes in prostate cancer. Science 310: 644-648.

Tomlins S A, Mehra R, Rhodes D R, Cao X, Wang L, Dhanasekaran S M et al (2007). Integrative molecular concept modeling of prostate cancer progression. Nat Genet 39: 41-51.

Varambally S, Yu J, Laxman B, Rhodes D R, Mehra R, Tomlins S A et al (2005). Integrative genomic and proteomic analysis of prostate cancer reveals signatures of metastatic progression. Cancer Cell 8: 393-406.
Wang 1-1, Owens C. Chandra N, Conaway M R, Brautigan D L, Theodorescu D (2010). Phosphorylation of RalB is important for bladder cancer cell growth and metastasis. Cancer Res 70: 8760-8769.
Wang R, Morris D S, Tomlins S A, Lonigro R J, Tsodikov A, Mehra R et al (2009). Development of a Multiplex Quantitative PCR Signature to Predict Progression in Non-Muscle-Invasive Bladder Cancer. Cancer Res 69: 3810-3818.
Wang K, Chen Y, Liu S, Qiu S, Gao S, Huang X et al (2009). Immunogenicity of RalA and its tissue-specific expression in carcinoma. Int J Immunopathol Pharmacol 22: 735-743.
Ward Y, Wang W, Woodhouse E, Linnoila I, Liotta L, Kelly K (2001). Signal pathways which promote invasion and metastasis: critical and distinct contributions of extracellular signal regulated kinase and Ral-specific guanine exchange factor pathways. Mol Cell Biol 21: 5958-5969.
Wei Q, Li M, Fu X, Tang R, Na Y, Jiang M et al (2007). Global analysis of differentially expressed genes in androgen-independent prostate cancer. Prostate Cancer Prostatic Dis 10: 167-174.
Williams P D, Cheon S, Havaleshko D M, Jeong Fr, Cheng F, Theodorescu D et al (2009). Concordant gene expression signatures predict clinical outcomes of cancer patients undergoing systemic therapy. Cancer Res 69: 8302-8309.

Wood L D, Parsons D W, Jones S, Lin J, Sjoblom T, Leary R J et al (2007). The genomic landscapes of human breast and colorectal cancers. Science 318: 1108-1113.
Wu Z, Owens C, Chandra N, Popovic K, Conaway M, Theodorescu D (2010). RalBPI is necessary for metastasis of human cancer cell lines. Neoplasia 12: 1003-1012.
Ye H, Yu T, Temam S, Ziober B L, Wang J, Schwartz J L et al (2008). Transcriptomic dissection of tongue squamous cell carcinoma. BMC Genomics 9:69.
Yin J, Pollock C, Tracy K, Chock M, Martin P, Oberst M et al (2007): Activation of the RalGEF/Ral pathway promotes prostate cancer metastasis to bone. Mol Cell Biol 27: 7538-7550.

### TABLES

TABLE 1
RalA staining class versus clinicopathologic features

| | RalA Low | RalA High | P-Value* |
|---|---|---|---|
| pTa | 4 | 0 | 0.97 |
| pT1 | 3 | 2 | |
| pT2 | 30 | 17 | |
| pT3 | 44 | 19 | |
| pT4 | 17 | 7 | |
| pN0 | 71 | 28 | 0.22 |
| pN1+ | 18 | 12 | |
| Female | 23 | 16 | 0.13 |
| Male | 73 | 28 | |
| LVSI− | 57 | 26 | 0.80 |
| LVSI+ | 38 | 19 | |
| CIS− | 77 | 33 | 0.38 |
| CIS+ | 12 | 8 | |
| Urothelial Carcinoma | 79 | 30 | 0.028 |
| Squamous Cell Carcinoma | 14 | 7 | |
| Adenocarcinoma | 2 | 4 | |
| Small Cell Carcinoma | 0 | 3 | |
| Sarcomatoid | 3 | 1 | |

*Two-tailed P-value for Chi-Squared statistic against the assumption of independence

TABLE 2
RalB staining class versus clinicopathologic features

| | RalB Low | RalB High | P-Value |
|---|---|---|---|
| pTa | 2 | 1 | 0.64 |
| pT1 | 1 | 4 | |
| pT2 | 22 | 23 | |
| pT3 | 44 | 17 | |
| pT4 | 9 | 14 | |
| pN0 | 51 | 46 | 0.24 |
| pN1+ | 17 | 9 | |
| Female | 22 | 17 | 0.94 |
| Male | 56 | 42 | |
| LVSI− | 40 | 40 | 0.056 |
| LVSI+ | 36 | 18 | |
| CIS− | 61 | 43 | 0.91 |
| CIS+ | 12 | 8 | |
| Urothelial Carcinoma | 62 | 42 | 0.018 |
| Squamous Cell Carcinoma | 8 | 12 | |
| Adenocarcinoma | 1 | 5 | |
| Small Cell Carcinoma | 3 | 0 | |
| Sarcomatoid | 4 | 0 | |

* Two-tailed P-value for Chi-Squared statistic against the assumption of independence

TABLE 3
Association between staining for RalA and RalB

| | RalA Low | RalA High | |
|---|---|---|---|
| RalB Low | 57 | 20 | 0.1059 |
| RalB High | 36 | 23 | |

TABLE 4
Probsets Regulated 2-Fold by RalA and RalB

| Probeset* | Fold Change\hat{ } | HUGO Symbol |
|---|---|---|
| 210095_s_at | 4.97 | IGFBP3 |
| 222043_at | 4.88 | CLU |
| 201565_s_at | 4.63 | ID2 |
| 212143_s_at | 4.12 | IGFBP3 |
| 203325_s_at | 3.90 | COL5A1 |
| 221530_s_at | 3.83 | BHLHE41 |
| 204396_s_at | 3.73 | GRK5 |
| 204584_at | 3.73 | L1CAM |
| 212488_at | 3.56 | COL5A1 |
| 212489_at | 3.41 | COL5A1 |
| 213397_x_at | 3.23 | RNASE4 |
| 203845_at | 3.13 | KAT2B |
| 202196_s_at | 3.12 | DKK3 |
| 211071_s_at | 3.10 | MLLT11 |
| 206924_at | 3.09 | IL11 |
| 219410_at | 2.97 | TMEM45A |
| 205158_at | 2.80 | RNASE4 |
| 218625_at | 2.76 | NRN1 |
| 214247_s_at | 2.68 | DKK3 |
| 206117_at | 2.65 | TPM1 |
| 207469_s_at | 2.55 | PIR |
| 212888_at | 2.51 | DICER1 |
| 203743_s_at | 2.50 | TDG |
| 209135_at | 2.49 | ASPH |
| 202952_s_at | 2.42 | ADAM12 |
| 202733_at | 2.40 | P4HA2 |
| 210896_s_at | 2.39 | ASPH |
| 213790_at | 2.38 | ADAM12 |
| 202743_at | 2.35 | PIK3R3 |
| 205199_at | 2.35 | CA9 |
| 204341_at | 2.33 | TRIM16 |
| 213005_s_at | 2.33 | KANK1 |
| 201506_at | 2.33 | TGFBI |
| 221541_at | 2.30 | CRISPLD2 |
| 208792_s_at | 2.30 | CLU |
| 208791_at | 2.28 | CLU |
| 206116_s_at | 2.27 | TPM1 |
| 212099_at | 2.23 | RHOB |
| 222062_at | 2.21 | IL27RA |
| 209822_s_at | 2.19 | VLDLR |
| 210986_s_at | 2.17 | TPM1 |
| 201505_at | 2.17 | LAMB1 |
| 219888_at | 2.12 | SPAG4 |
| 203871_at | −2.12 | SENP3 |
| 211935_at | −2.26 | ARL6IP1 |
| 218190_s_at | −2.27 | UQCR10 |
| 208756_at | −2.31 | EIF3I |
| 212150_at | −2.49 | EFR3A |
| 201087_at | −2.57 | PXN |
| 211823_s_at | −2.72 | PXN |
| 215113_s_at | −2.74 | SENP3 |
| 213524_s_at | −2.76 | G0S2 |
| 207850_at | −2.77 | CXCL3 |
| 221263_s_at | −2.84 | SF3B5 |
| 209774_x_at | −2.88 | CXCL2 |
| 215171_s_at | −3.04 | TIMM17A |
| 212149_at | −3.19 | EFR3A |
| 201529_s_at | −3.32 | RPA1 |
| 201528_at | −3.48 | RPA1 |
| 204475_at | −5.05 | MMP1 |

*Affymetrix HG-U133A
\hat{ }Fold change, on average across duplicate RalA and RalB-depleted replicates, relative to replicate siControl duplicates.

TABLE 5
COXEN Concordant Probsets Regulated 2-Fold by RalA and RalB

| Probeset* | Fold Change\hat{ } | HUGO Symbol |
|---|---|---|
| 222043_at | 4.88 | CLU |
| 203325_s_at | 3.90 | COL5A1 |
| 204396_s_at | 3.73 | GRK5 |
| 204584_at | 3.73 | L1CAM |
| 212488_at | 3.56 | COL5A1 |
| 212489_at | 3.41 | COL5A1 |
| 213397_x_at | 3.23 | RNASE4 |
| 203845_at | 3.13 | KAT2B |
| 202196_s_at | 3.12 | DKK3 |
| 211071_s_at | 3.10 | MLLT11 |
| 206924_at | 3.09 | IL11 |
| 205158_at | 2.80 | RNASE4 |
| 218625_at | 2.76 | NRN1 |
| 214247_s_at | 2.68 | DKK3 |
| 206117_at | 2.65 | TPM1 |
| 212888_at | 2.51 | DICER1 |
| 202952_s_at | 2.42 | ADAM12 |
| 202733_at | 2.40 | P4HA2 |
| 213790_at | 2.38 | ADAM12 |
| 202743_at | 2.35 | PIK3R3 |
| 213005_s_at | 2.33 | KANK1 |
| 201506_at | 2.33 | TGFBI |
| 221541_at | 2.30 | CRISPLD2 |
| 208792_s_at | 2.30 | CLU |
| 208791_at | 2.28 | CLU |
| 206116_s_at | 2.27 | TPM1 |
| 212099_at | 2.23 | RHOB |
| 222062_at | 2.21 | IL27RA |
| 209822_s_at | 2.19 | VLDLR |
| 210986_s_at | 2.17 | TPM1 |
| 201505_at | 2.17 | LAMB1 |
| 203871_at | −2.12 | SENP3 |
| 218190_s_at | −2.27 | UQCR10 |
| 208756_at | −2.31 | EIF3I |
| 215113_s_at | −2.74 | SENP3 |
| 221263_s_at | −2.84 | SF3B5 |
| 215171_s_at | −3.04 | TIMM17A |
| 201528_at | −3.48 | RPA1 |
| 204475_at | −5.05 | MMP1 |

*Affymetrix HG-U133A
\hat{ }Fold change, on average across duplicate RalA and RalB-depleted replicates, relative to replicate siControl duplicates.

What is claimed is:

1. A method for monitoring the progression of cancer in a subject, the method comprising:
a) measuring the expression level of a plurality of markers in a first biological sample obtained from the subject, wherein the plurality of markers comprise a plurality of markers selected from the group consisting of:
i) a marker gene having at least 95% sequence identity with a gene selected from the Table 5, or homologs or variants thereof;
ii) polypeptides encoded by the marker genes of i)
iii) fragments of polypeptides of ii); and
iv) a polynucleotide which is fully complementary to at least a portion of a marker gene of i);
b) measuring the expression level of the plurality of markers in a second biological sample obtained from the subject; and
c) comparing the expression level of the markers measured in the first sample with the level of the markers measured in the second sample.

2. The method of claim 1, wherein the genes detected share 100% sequence identity with the corresponding marker gene in i).

3. The method of claim 1, wherein the first biological sample from the subject is obtained at a time t₀, and the second biological sample from the subject is obtained at a later time t₁.

4. (canceled)

5. The method of claim 1, wherein a level of at least one of the plurality of markers is determined and compared to a standard level or reference range.

6. The method of claim 1, wherein the presence of the marker is determined by detecting the presence of a polypeptide.

7. The method of claim 6, wherein the method further comprises detecting the presence of the polypeptide using a reagent that specifically binds to the polypeptide or a fragment thereof.

8. The method of claim 7, wherein the reagent is selected from the group consisting of an antibody, an antibody derivative, and an antibody fragment.

9. The method of claim 1, wherein the presence of the marker is determined by obtaining RNA from the cancer tissue sample; generating cDNA from the RNA; amplifying the cDNA with probes or primers for marker genes; obtaining from the amplified cDNA the expression levels of the genes or gene expression products in the sample.

10. The method of claim 1, wherein the patient is a human.

11. The method of claim 1, wherein the cancer is selected from the group consisting of bladder cancer, prostate cancer and squamous cell carcinoma.

12. A method of assessing the efficacy of a treatment for cancer in a subject, the method comprising comparing:
a) the expression level of a plurality of markers measured in a first sample obtained from the subject at a time t₀, wherein the plurality of markers comprise a plurality of markers selected from the group consisting of:
i) a marker gene having at least 95% sequence identity with a gene selected from Table 5, or homologs or variants thereof;
ii) polypeptides encoded by the marker genes of i);
iii) fragments of polypeptides of ii); and
iv) a polynucleotide which is fully complementary to at least a portion of a marker gene of i); and
b) the level of the plurality of markers in a second sample obtained from the subject at time t₁;
wherein a change in the levels of the markers in the second sample relative to the first sample is an indication that the treatment is efficacious for treating cancer in the subject.

13. (canceled)

14. The method of claim 12, wherein the time t₀ is before the treatment has been administered to the subject, and the time t₁ is after the treatment has been administered to the subject.

15. (canceled)

16. The method of claim 1, wherein the cancer is selected from the group consisting of bladder cancer, prostate cancer and squamous cell carcinoma.

17. An assay system for predicting patient response or outcome to anti-cancer therapy comprising a means to detect:
a) the expression of a plurality of marker genes selected from the group consisting of:
i) a marker gene having at least 95% sequence identity with a gene selected from Table 5, or homologs or variants thereof;
ii) polypeptides encoded by the marker genes of i);
iii) fragments of polypeptides of ii); and
iv) a polynucleotide which is fully complementary to at least a portion of a marker gene of i).

18. The assay system of claim 17, wherein the means to detect comprises nucleic acid probes comprising at least 10 to 50 contiguous nucleic acids of the marker gene(s), or complementary nucleic acid sequences thereof.

19. The assay system of claim 17, wherein the means to detect comprises binding ligands that specifically detect polypeptides encoded by the marker genes.

20. (canceled)

21. The assay system of claim 17, wherein the means to detect comprises at least one of nucleic acid probes and binding ligands disposed on an assay surface.

22. The assay system of claim 22, wherein the assay surface comprises a chip, array, or fluidity card.

23. (canceled)

24. The assay system of claim 21, wherein the binding ligands comprise antibodies or binding fragments thereof.

25. The assay system of claim 17, further comprising: a control selected from the group consisting of:
a) information containing a predetermined control level of the marker gene that has been correlated with response to the administration of a therapeutic treatment; and
b) information containing a predetermined control level of the marker gene that has been correlated with a lack of response to the administration of a therapeutic treatment.

26. (canceled)
